(19) United States
(12) Patent Application Publication
(10) Pub. No.: US 2016/0346398 A1
(43) Pub. Date: Dec. 1, 2016

(54) CROSS-LINKED HYALURONIC ACID FOR DRUG DELIVERY AND PHARMACEUTICAL PREPARATION USING SAME

(71) Applicant: ALURON BIOPHARMA INC., Montreal (CA)

(72) Inventors: Mikhail SELYANIN, Moscow (RU); Felix POLYAK, Montreal (CA); Kirill SHINGEL, Brossard (CA); Nataliya MYKHAYLOVA, Moscow (RU)

(21) Appl. No.: 15/168,763

(22) Filed: May 31, 2016

Related U.S. Application Data
(60) Provisional application No. 62/168,411, filed on May 29, 2015.

(51) Int. Cl. A61K 47/48; A61K 31/4535; A61K 31/4196; A61K 31/4745

(52) U.S. Cl. CPC A61K 47/48092; A61K 47/48784; A61K 31/4745; A61K 31/4535; A61K 31/4196

(57) ABSTRACT

A water-soluble gel polymer matrix comprising hyaluronic acid and an antitumor agent is described herein. The antitumor agent is crosslinked with the hyaluronic acid by at least one of covalent and/or non-covalent bonding resulting in improved water solubility of the antitumor agent. Pharmacokinetic studies illustrated that the antitumor agent exhibits enhanced Cₘₐₓ (maximum drug plasma concentration) and Tₘₐₓ (time required to reach Cₘₐₓ) values.

### CROSS REFERENCE TO RELATED APPLICATIONS

This application claims the benefit of U.S. Provisional Application 62/168,411, filed May 29, 2015. The contents of the referenced application are incorporated into the present application by reference.

### FIELD

The present disclosure broadly relates to a drug delivery vehicle for cancer therapy, a process for producing the same, and a pharmaceutical preparation using the same. More specifically, but not exclusively, the present disclosure relates to cross-linked hyaluronic acid for delivering anti-cancer agents, methods for their preparation and pharmaceutical preparations using same. The anti-cancer agents can be cross-linked with the hyaluronic acid.

### BACKGROUND

Hyaluronic acid (HA) is a naturally occurring polyanionic, non-sulfated glycosaminoglycan that consists of N-acetyl-D-glucosamine and β-glucoronic acid. It is present in the intercellular matrix of most vertebrate connective tissues especially skin and joints where it has a protective, structure stabilizing and shock-absorbing role. Hyaluronic acid is highly soluble in its natural state and has a rapid turnover through enzymatic and free radical metabolization.

The unique viscoelastic nature of HA along with its biocompatibility and non-immunogenicity has led to its use in a number of clinical applications, which include: the supplementation of joint fluid in arthritis; as a surgical aid in eye surgery; and to facilitate the healing and regeneration of surgical wounds. More recently, HA has been investigated as a drug delivery agent for various routes of administration, including ophthalmic, nasal, pulmonary, parenteral, and topical.

The delivery of a drug to a patient with controlled-release of the active ingredient has been an active area of research for decades and has been fueled by the many recent developments in polymer science and the need to deliver more labile pharmaceutical agents such as small molecule drugs, nucleic acids, proteins, and peptides. Biodegradable particles have been developed as sustained release vehicles used in the administration of small molecule drugs as well as protein and peptide drugs and nucleic acids. The drugs are typically encapsulated in a polymer matrix which is biodegradable and biocompatible. As the polymer is degraded and/or as the drug diffuses out of the polymer, the drug is released into the body. Typical polymers used in preparing these particles are polyesters such as poly(glycolide-co-lactide) (PLGA), polyglycolic acid, poly-p-hydroxybutyrate, and polyacrylic acid ester. These particles have the additional advantage of protecting the drug from degradation by the body. These particles, depending on their size, composition, and the drug being delivered can be administered to an individual using any route available.

Biocompatibility is of special importance when a sustained release vehicle is used for targeted delivery of a drug, particularly if the dwell time of the vehicle is much longer than the clinical efficacy of the delivered drug. Controlled release technology can prolong the effect of the drug and improve the therapeutic index, and therefore lends itself naturally to the problem of providing prolonged duration of action.

### SUMMARY

The present disclosure broadly relates to hyaluronic acid-based drug delivery vehicles for cancer therapy. The drug delivery vehicles can include an anticancer/antitumor drug that has been crosslinked with hyaluronic acid through covalent, ionic, and/or electrostatic bonding. This can result in a crosslinked hyaluronic acid matrix where the drug serves a dual purpose by acting as a cross-linker as well as a therapeutic. Without wishing to be bound by theory, and as described and illustrated in non-limiting embodiments throughout this disclosure, such a drug delivery vehicle can provide several benefits ranging from: (1) increased solubility and stability of the drug; (2) targeted specificity and/or increased selectivity of the drug delivery vehicle to cells exhibiting pathologic activities of cancer cells (e.g., overexpression of CD44 in cancer cells); and/or (3) reduced drug dosage amounts. Stated another way, the drug delivery vehicles can stabilize and solubilize the drug while also reducing the severe toxic side effects that are typically associated with anticancer drugs on healthy tissues. This can be achieved through lower dosages of the drug, thereby increasing the cost efficiency of cancer treatment while ameliorating the potential side effects typically associated with anticancer drugs.

Accordingly, in an embodiment, the present disclosure broadly relates to a delivery system for delivering an intended drug specifically to a desired cell or tissue.

The present disclosure relates to cross-linked hyaluronic acid as a drug delivery vehicle for cancer therapy. In an embodiment, the hyaluronic acid is cross-linked using a biologically active compound. In a further embodiment, the biologically active compound is a prophylactic and/or therapeutic agent. In yet a further embodiment, the biologically active compound is an anti-tumor agent.

In an embodiment, the present disclosure relates to a pharmaceutical preparation comprising a drug enclosed or encapsulated within the drug delivery vehicle for cancer therapy. In a further embodiment, the drug is a small molecular compound such as an antitumor agent. In a further embodiment, the antitumor compound can be at least one member selected from the group consisting of azacitidine, imatinib, lenalidomide, etoposide, topotecan, irinotecan, letrozole, raloxifene, cyclophosphamide, mechlorethamine, carbazylquinone, melphalan, thiotepa, busulfan, nimustine, carmustine, procarbazine, dacarbazine, methotrexate, 6-mercaptopurine, 6-thioguanine, azathioprine, 5-fluorouracil, ftorafur, floxuridine, cytarabine, ancitabine, doxifluridine, actinomycinD, bleomycin, mitomycin, chromomycin A3, cinelbin A, aclacinomycin A, adriamycin, peplomycin, cisplatin, mitoxantrone, epirubicin, pirarubicin, vinblastine, vincristine, vindesine, carboplatin, estramustine phosphate, mitotane, porphyrin, paclitaxel and docetaxel. In still other aspects, any combination of these antitumor compounds can be incorporated into a drug delivery vehicle of the present disclosure. In a further embodiment, the antitumor compound can be a boron-containing compound. The boron-containing compound can be mercaptoundecahydrododecaborate (BSH) or p-boronophenylalanine (BPA). In yet a further embodiment of the present disclosure, the pharmaceutical preparation containing the boron-containing compound can be used in boron neutron capture therapy (BNCT).

In an embodiment, the pharmaceutical preparation can be used in therapy of one member selected from sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma. The pharmaceutical preparations of the present disclosure are also applicable to sarcomas and epithelial cancers, such as ovarian cancers and breast cancers.

In an embodiment, the present disclosure relates to compositions comprising cross-linked hyaluronic acid providing for prolonged action.

In an embodiment, the present disclosure relates to hyaluronic acid compositions wherein the hyaluronic acid is cross-linked using a small molecular compound such as an antitumor agent.

In an embodiment, the present disclosure relates to hyaluronic acid compositions wherein the hyaluronic acid is cross-linked using one or more antitumor agents providing for at least one of covalent, ionic, and/or electrostatic (e.g. H-bonding) interactions with the hyaluronic acid. In an embodiment of the present disclosure, the antitumor agents comprise one or more functionalities providing for at least one of covalent, ionic, and/or electrostatic (e.g. H-bonding) interactions with the hyaluronic acid. Non-limiting examples of such functionalities include amine groups, hydroxyl groups and carbonyl containing functionalities.

In an embodiment, the present disclosure relates to hyaluronic acid compositions, wherein the hyaluronic acid is cross-linked using antitumor agents providing for non-covalent interactions with the hyaluronic acid.

In an embodiment, the present disclosure relates to crosslinked hyaluronic acid compositions for use in the targeted delivery of biologically active compounds to a desired cell or tissue. In an embodiment, the biologically active compound is an anti-tumor agent.

In an embodiment, the present disclosure relates to crosslinked hyaluronic acid compositions for use in cancer therapy. The type of cancer to be treated determines the biologically active compound encapsulated within the hyaluronic acid matrix. Non-limiting examples of cancers include sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma. The crosslinked hyaluronic acid compositions are also for use in therapy of sarcomas and epithelial cancers, such as ovarian cancers and breast cancers.

There is a strong demand for a hyaluronic acid composition that regulates in vivo behavior of an intended drug and delivers the drug efficiently, specifically and easily to a target such as a cell or tissue.

In an embodiment, the present disclosure relates to a process for making a cross-linked hyaluronic acid, the process comprising mixing hyaluronic acid with at least one biologically active compound to produce a mixture; and feeding the mixture into an extruder to produce the cross-linked hyaluronic acid. In an embodiment, the biologically active compound is an anti-tumor agent.

In an embodiment, the present disclosure relates to a method for treating a cancer comprising administering a composition comprising a cross-linked hyaluronic acid to a subject in need of treatment. Non-limiting examples of cancers include sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma as well as sarcomas and epithelial cancers, such as ovarian cancers and breast cancers.

In an embodiment, the present disclosure relates to hyaluronic acid compositions for cancer drug therapy that has lower adverse side effects on healthy cells/organs, prolonged therapeutic activity and better efficacy.

In an embodiment, the present disclosure relates to a water-soluble gel polymer matrix comprising hyaluronic acid having a molecular weight between 10,000 Da and 7,000,000 Da and an antitumor agent. The antitumor agent is crosslinked with the polymer matrix. This crosslinking with the polymer matrix improves the water solubility of the antitumor agent when compared to the antitumor agent itself. In an aspect of the present disclosure, the antitumor agent of the water-soluble gel polymer matrix exhibits enhanced Cₘₐₓ (maximum drug plasma concentration) and Tₘₐₓ (time required to reach Cₘₐₓ) values. In an aspect of the present disclosure, the antitumor agent is crosslinked by at least one of covalent and/or electrostatic bonding. In an aspect of the present disclosure, the crosslinking is achieved by an extrusion process.

In an embodiment, the present disclosure relates to a pharmaceutical delivery vehicle comprising hyaluronic acid having a molecular weight between 10,000 Da and 7,000,000 Da and an antitumor agent. The antitumor agent is crosslinked with the hyaluronic acid to form a water-soluble gel polymer matrix. This crosslinking with the polymer matrix improves the water solubility of the antitumor agent when compared to the antitumor agent itself. In an aspect of the present disclosure, the antitumor agent of the pharmaceutical delivery vehicle exhibits enhanced Cₘₐₓ (maximum drug plasma concentration) and Tₘₐₓ (time required to reach Cₘₐₓ) values. In an aspect of the present disclosure, the antitumor agent is crosslinked by at least one of covalent and/or electrostatic bonding. In an aspect of the present disclosure, the crosslinking is achieved by an extrusion process.

In an embodiment, the present disclosure relates to process for preparing a crosslinked hyaluronic acid matrix. The process can include extruding hyaluronic acid to produce extruded hyaluronic acid, mixing the extruded hyaluronic acid with an antitumor agent to produce a mixture, and extruding the mixture to produce the crosslinked hyaluronic acid matrix.

Also disclosed in the context of the present disclosure are embodiments 1 to 30. Embodiment 1 is a water-soluble gel polymer matrix comprising: hyaluronic acid having a molecular weight between 10,000 Da and 7,000,000 Da; and an antitumor agent, wherein the antitumor agent is crosslinked with the polymer matrix, and wherein the polymer matrix improves the water solubility of the antitumor agent. Embodiment 2 is the water-soluble gel polymer matrix of embodiment 1, wherein the antitumor agent is crosslinked by at least one of covalent and/or electrostatic bonding. Embodiment 3 is the water-soluble gel polymer matrix of embodiment 2, wherein the antitumor agent is crosslinked by electrostatic bonding, and wherein the electrostatic bonding is hydrogen bonding. Embodiment 4 is the water-soluble gel polymer matrix of embodiment 3, wherein the antitumor agent is crosslinked by covalent bonding. Embodiment 5 is the water-soluble gel polymer matrix of any one of embodiments 1 to 4, wherein the antitumor agent is selected from azacitidine, imatinib, lenalidomide, etoposide, topotecan, irinotecan, letrozole, raloxifene, cyclophosphamide, mechlorethamine, carbazylquinone, melphalan, thiotepa, busulfan, nimustine, carmustine, procarbazine, dacarbazine, methotrexate, 6-mercaptopurine, 6-thioguanine, azathioprine, 5-fluorouracil, ftorafur, floxuridine, cytarabine, ancitabine, doxifluridine, actinomycinD, bleomycin, mitomycin, chromomycin A3, cinelbin A, aclacinomycin A, adriamycin, peplomycin, cisplatin, mitoxantrone, epirubicin, pirarubicin, vinblastine, vincristine, vindesine, carboplatin, estramustine phosphate, mitotane, porphyrin, paclitaxel and docetaxel. Embodiment 6 is the water-soluble gel polymer matrix of any one of embodiments 1 to 5, wherein the ratio of the hyaluronic acid to antitumor agent is from about 20:1 to about 2:1. Embodiment 7 is the water-soluble gel polymer matrix of embodiment 6, wherein the ratio of the hyaluronic acid to antitumor agent is from about 10:1 to about 2:1. Embodiment 8 is the water-soluble gel polymer matrix of embodiment 7, wherein the ratio of the hyaluronic acid to antitumor agent is from about 5:1 to about 2:1. Embodiment 9 is the water-soluble gel polymer matrix of any one of embodiments 1 to 8, wherein the crosslinking is achieved by an extrusion process. Embodiment 10 is the water-soluble gel polymer matrix of any one of embodiments 1 to 9, wherein the antitumor agent exhibits enhanced Cₘₐₓ (maximum drug plasma concentration) and Tₘₐₓ (time required to reach Cₘₐₓ) values.

Embodiment 11 is a pharmaceutical delivery vehicle comprising: hyaluronic acid having a molecular weight between 10,000 Da and 7,000,000 Da; and an antitumor agent; wherein the antitumor agent is crosslinked with the hyaluronic acid to form a water-soluble gel polymer matrix and wherein the polymer matrix improves the water solubility of the antitumor agent. Embodiment 12 is the pharmaceutical delivery vehicle of embodiment 11, wherein the antitumor agent is crosslinked by at least one of covalent and/or electrostatic bonding. Embodiment 13 is the pharmaceutical delivery vehicle of embodiment 12, wherein the antitumor agent is crosslinked by electrostatic bonding, and wherein the electrostatic bonding is hydrogen bonding. Embodiment 14 is the pharmaceutical delivery vehicle of embodiment 12, wherein the antitumor agent is crosslinked by covalent bonding. Embodiment 15 is the pharmaceutical delivery vehicle of any one of embodiments 11 to 14, wherein the antitumor agent is selected from azacitidine, imatinib, lenalidomide, etoposide, topotecan, irinotecan, letrozole, raloxifene, cyclophosphamide, mechlorethamine, carbazylquinone, melphalan, thiotepa, busulfan, nimustine, carmustine, procarbazine, dacarbazine, methotrexate, 6-mercaptopurine, 6-thioguanine, azathioprine, 5-fluorouracil, ftorafur, floxuridine, cytarabine, ancitabine, doxifluridine, actinomycinD, bleomycin, mitomycin, chromomycin A3, cinelbin A, aclacinomycin A, adriamycin, peplomycin, cisplatin, mitoxantrone, epirubicin, pirarubicin, vinblastine, vincristine, vindesine, carboplatin, estramustine phosphate, mitotane, porphyrin, paclitaxel and docetaxel. Embodiment 16 is the pharmaceutical delivery vehicle of any one of embodiments 11 to 15, wherein the ratio of the hyaluronic acid to antitumor agent is from about 20:1 to about 2:1. Embodiment 17 is the pharmaceutical delivery vehicle of embodiment 16, wherein the ratio of the hyaluronic acid to antitumor agent is from about 10:1 to about 2:1. Embodiment 18 is the pharmaceutical delivery vehicle of embodiment 17, wherein the ratio of the hyaluronic acid to antitumor agent is from about 5:1 to about 2:1. Embodiment 19 is the pharmaceutical delivery vehicle of any one of embodiments 11 to 18, wherein the crosslinking is achieved by an extrusion process. Embodiment 20 is the pharmaceutical delivery vehicle of any one of embodiments 11 to 19, wherein the antitumor agent exhibits enhanced Cₘₐₓ (maximum drug plasma concentration) and Tₘₐₓ (time required to reach Cₘₐₓ) values.

Embodiment 21 is a process for preparing a crosslinked hyaluronic acid matrix, the method comprising: extruding hyaluronic acid to produce extruded hyaluronic acid; mixing the extruded hyaluronic acid with an antitumor agent to produce a mixture; and extruding the mixture to produce the crosslinked hyaluronic acid matrix. Embodiment 22 is the process of embodiment 21, further comprising mixing the crosslinked hyaluronic acid matrix with additional hyaluronic acid and extruding. Embodiment 23 is the process of embodiment 21 or 22, wherein the hyaluronic acid has a molecular weight between 10,000 Da and 7,000,000 Da. Embodiment 24 is the process of any one of embodiments 21 to 23, wherein the antitumor agent is selected from azacitidine, imatinib, lenalidomide, etoposide, topotecan, irinotecan, letrozole, raloxifene, cyclophosphamide, mechlorethamine, carbazylquinone, melphalan, thiotepa, busulfan, nimustine, carmustine, procarbazine, dacarbazine, methotrexate, 6-mercaptopurine, 6-thioguanine, azathioprine, 5-fluorouracil, ftorafur, floxuridine, cytarabine, ancitabine, doxifluridine, actinomycinD, bleomycin, mitomycin, chromomycin A3, cinelbin A, aclacinomycin A, adriamycin, peplomycin, cisplatin, mitoxantrone, epirubicin, pirarubicin, vinblastine, vincristine, vindesine, carboplatin, estramustine phosphate, mitotane, porphyrin, paclitaxel and docetaxel. Embodiment 25 is the process of any one of embodiments 21 to 24, wherein the ratio of the hyaluronic acid to antitumor agent is from about 20:1 to about 2:1. Embodiment 26 is the process of embodiment 25, wherein the ratio of the hyaluronic acid to antitumor agent is from about 10:1 to about 2:1. Embodiment 27 is the process of embodiment 26, wherein the ratio of the hyaluronic acid to antitumor agent is from about 5:1 to about 2:1. Embodiment 28 is the process of any one of embodiments 21 to 27, wherein the antitumor agent is crosslinked by covalent and/or electrostatic bonding. Embodiment 29 is the process of embodiment 28, wherein the antitumor agent is crosslinked by electrostatic bonding, and wherein the electrostatic bonding is hydrogen bonding. Embodiment 30 is the process of embodiment 29, wherein the antitumor agent is crosslinked by covalent bonding.

The foregoing and other advantages and features of the present disclosure will become more apparent upon reading of the following non-restrictive description of illustrative embodiments thereof, given by way of example with reference to the accompanying drawings/figures.

### BRIEF DESCRIPTION OF THE DRAWINGS/FIGURES

In the appended drawings/figures:

FIG. 1 illustrates the effect of a hyaluronic acid composition comprising Azacitidine (Vidaza™) on the MM.1S cell line, following a 2-day incubation period; NC—negative control (hyaluronic acid); PC—positive control (pure drug); ABP—complex drug-HA; Mix—mechanical mixture, no extrusion.

FIG. 2 illustrates the effect of a hyaluronic acid composition comprising Azacitidine on the MM.1S cell line, following a 3-day incubation period; NC—negative control (hyaluronic acid); PC—positive control (pure drug); ABP—complex drug-HA; Mix—mechanical mixture, no extrusion.

FIG. 3 illustrates the effect of a hyaluronic acid composition comprising Imatinib (Gleevec™) on the K-562 cell line, following a 2-day incubation period; NC—negative control (hyaluronic acid); PC—positive control (pure drug); ABP—complex drug-HA; Mix—mechanical mixture, no extrusion.

FIG. 4 illustrates the effect of a hyaluronic acid composition comprising Imatinib on the K-562 cell line, following a 3-day incubation period; NC—negative control (hyaluronic acid); PC—positive control (pure drug); ABP—complex drug-HA; Mix—mechanical mixture, no extrusion.

FIG. 5 illustrates the effect of a hyaluronic acid composition comprising Lenalidomide (Revlimid™) on the MM.1S cell line, following a 2-day incubation period; NC—negative control (hyaluronic acid); PC—positive control (pure drug); ABP—complex drug-HA; Mix—mechanical mixture, no extrusion.

FIG. 6 illustrates the effect of a hyaluronic acid composition comprising Lenalidomide on the MM.1S cell line, following a 3-day incubation period; NC—negative control (hyaluronic acid); PC—positive control (pure drug); ABP—complex drug-HA; Mix—mechanical mixture, no extrusion.

FIG. 7 illustrates the effect of a hyaluronic acid composition comprising Etoposide (Etopophos™) on the HL-60 cell line, following a 2-day incubation period; NC—negative control (hyaluronic acid); PC—positive control (pure drug); ABP—complex drug-HA; Mix—mechanical mixture, no extrusion.

FIG. 8 illustrates the effect of a hyaluronic acid composition comprising Etoposide on the HL-60 cell line, following a 3-day incubation period; NC—negative control (hyaluronic acid); PC—positive control (pure drug); ABP—complex drug-HA; Mix—mechanical mixture, no extrusion.

FIG. 9 illustrates the effect of a hyaluronic acid composition comprising Topotecan (Hycamtin™) on the HCT-116 cell line, following a 2-day incubation period; NC—negative control (hyaluronic acid); PC—positive control (pure drug); ABP—complex drug-HA; Mix—mechanical mixture, no extrusion.

FIG. 10 illustrates the effect of a hyaluronic acid composition comprising Topotecan on the HCT-116 cell line, following a 3-day incubation period; NC—negative control (hyaluronic acid); PC—positive control (pure drug); ABP—complex drug-HA; Mix—mechanical mixture, no extrusion.

FIG. 11 illustrates the effect of a hyaluronic acid composition comprising Irinotecan (Camptosar™) on the HCT-116 cell line, following a 2-day incubation period; NC—negative control (hyaluronic acid); PC—positive control (pure drug); ABP—complex drug-HA; Mix—mechanical mixture, no extrusion.

FIG. 12 illustrates the effect of a hyaluronic acid composition comprising Irinotecan on the HCT-116 cell line, following a 3-day incubation period; NC—negative control (hyaluronic acid); PC—positive control (pure drug); ABP—complex drug-HA; Mix—mechanical mixture, no extrusion.

FIG. 13 illustrates the effect of a hyaluronic acid composition comprising Letrozole (Femara™) on the MCF-7 cell line, following a 2-day incubation period; NC—negative control (hyaluronic acid); PC—positive control (pure drug); ABP—complex drug-HA; Mix—mechanical mixture, no extrusion.

FIG. 14 illustrates the effect of a hyaluronic acid composition comprising Letrozole on the MCF-7 cell line, following a 3-day incubation period; NC—negative control (hyaluronic acid); PC—positive control (pure drug); ABP—complex drug-HA; Mix—mechanical mixture, no extrusion.

FIG. 15 illustrates the effect of a hyaluronic acid composition comprising Raloxifene (Evista™) on the MCF-7 cell line, following a 2-day incubation period; NC—negative control (hyaluronic acid); PC—positive control (pure drug); ABP—complex drug-HA; Mix—mechanical mixture, no extrusion.

FIG. 16 illustrates the effect of a hyaluronic acid composition comprising Raloxifene on the MCF-7 cell line, following a 3-day incubation period; NC—negative control (hyaluronic acid); PC—positive control (pure drug); ABP—complex drug-HA; Mix—mechanical mixture, no extrusion.

FIG. 17 illustrates the mean tumor volume over time in the vehicle group and treated animals between days 0 to 60 following tumor implementation. The tumor volume over time in the vehicle group and treated animals for human breast MCF-7 cancer tumor model without estrogen supplementation is represented as a mean tumor volume for each treatment group.

FIG. 18 illustrates the mean tumor volume over time in the vehicle group and treated animals between days 0 to 49 following tumor implementation. The tumor volume over time in the vehicle group and treated animals for human breast MCF-7 cancer tumor model with estrogen supplementation is represented as a mean tumor volume for each treatment group.

FIG. 19 illustrates the pharmacokinetics of Letrozole in rat blood plasma after oral administration of pure drug (5 mg/kg), and HA-Letrozole complexes (equivalent to 2 and 5 mg/kg of drug).

FIG. 20 illustrates the pharmacokinetics of Raloxifene in rat blood plasma after oral administration of pure drug (50 mg/kg), and HA-Raloxifene complex (equivalent to 50 and 15 mg/kg of drug).

### DETAILED DESCRIPTION

### Glossary

In order to provide a clear and consistent understanding of the terms and phrases used in the present disclosure, a number of definitions are provided below. Moreover, unless defined otherwise, all technical and scientific terms as used herein have the same meaning as commonly understood by one of ordinary skill in the art to which this specification pertains.

The word “a” or “an” when used in conjunction with the term “comprising” in the claims and/or the specification may mean “one”, but it is also consistent with the meaning of “one or more”, “at least one”, and “one or more than one” unless the content clearly dictates otherwise. Similarly, the word “another” may mean at least a second or more unless the content clearly dictates otherwise.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

As used in this specification and claim(s), the word “consisting” and its derivatives, are intended to be close ended terms that specify the presence of stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps. In one non-limiting aspect, a basic and novel characteristic of the present disclosure is the crosslinking of an antitumor agent with hyaluronic acid, which can improve/increase the water solubility of the antitumor agent.

The terms “about”, “substantially” and “approximately” as used herein mean a reasonable amount of deviation of the modified term such that the end result is not significantly changed. These terms of degree should be construed as including a deviation of at least ±1% of the modified term if this deviation would not negate the meaning of the word it modifies.

The term “extruder”, as used herein, is intended to refer to any conventional single or double screw extrusion device.

The term “residence time” in an extruder refers to the time taken by a material to get through the extruder, from the feed port to the die. The residence time is measured by adding a small quantity of material containing a coloring agent into the feed port. The chronometer is started when the colorant enters the barrel and is stopped when coloration is observed at the die exit.

The term “extrudate temperature” refers to the temperature of the material at the die exit of an extruder as measured by a portable thermocouple plunged into one of the die openings.

The term “cancer”, as used herein shall be given its ordinary meaning and is a general term for diseases in which abnormal cells divide without control. Cancer cells can invade nearby tissues and can spread through the bloodstream and lymphatic system to other parts of the body. There are several main types of cancer, for example, carcinoma is cancer that begins in the skin or in tissues that line or cover internal organs. Sarcoma is cancer that begins in bone, cartilage, fat, muscle, blood vessels, or other connective or supportive tissue. Leukemia is cancer that starts in blood-forming tissue such as the bone marrow, and causes large numbers of abnormal blood cells to be produced and enter the bloodstream. Lymphoma is cancer that begins in the cells of the immune system.

The terms “effective amount” and therapeutically-effective amount” as used herein means that amount of a compound, material, or composition comprising a compound or composition of the present disclosure, and which is effective for producing a desired therapeutic effect, biological or medicinal response in a tissue system, animal or human that is being sought by a researcher, veterinarian, medical doctor or other clinician, which includes alleviation of the symptoms of the disease or disorder being treated.

The phrase “pharmaceutically acceptable” is employed herein to refer to those compounds, materials, compositions, and/or dosage forms that are, within the scope of sound medical judgment, suitable for use in contact with the tissues of human beings and animals without excessive toxicity, irritation, allergic response, or other problem or complication, commensurate with a reasonable benefit/risk ratio.

The phrase “small molecule” as used herein refers to organic compounds, and salts thereof, whether naturally-occurring or artificially created (e.g., via chemical synthesis) that have relatively low molecular weight and that are not proteins, polypeptides, or nucleic acids. Typically, small molecules have a molecular weight of less than about 1500 g/mol. Also, small molecules typically have multiple carbon-carbon bonds. Known naturally-occurring small molecules include, but are not limited to, penicillin, erythromycin, taxol and rapamycin. Known synthetic small molecules include, but are not limited to, ampicillin, methicillin, sulfamethoxazole and sulfonamides.

As used herein, the term “stabilizing” includes maintaining a compound in a specific state and preventing or slowing fluctuations from that particular state into another.

As used herein, the term “biologically active” refers to the ability to mediate a biological function.

As used herein, the terms “cross-linking agent” and “cross-linker” are intended to cover a chemical agent that could react with hyaluronic acid through at least one of covalent and/or non-covalent bonds. Non-limiting examples of non-covalent bonds include ionic bonds, hydrophobic interactions, hydrogen bonds and van der Waals forces (dispersion attractions, dipole-dipole and dipole-induced interactions). In an embodiment of the present disclosure, the crosslinking agent is an antitumor agent.

The term “cross-linked” as used herein is intended to refer to two or more polymer chains of hyaluronic acid which have been covalently and/or non-covalently bonded via a cross-linking agent. Such cross-linking is differentiated from intermolecular or intramolecular dehydration which results in lactone, anhydride, or ester formation within a single polymer chain or between two or more chains. Although, it is contemplated that intramolecular cross-linking may also occur in the compositions as described herein. Cross-linking agents contain at least two functional groups that create covalent and/or non-covalent bonds between two or more molecules (i.e. hyaluronic acid chains). In an aspect of the present disclosure, the cross-linking agents comprise complimentary functional groups to that of hyaluronic acid such that the cross-linking can proceed. In an embodiment of the present disclosure, the crosslinking agent is an antitumor agent.

As used herein, the term “subject” means a human or animal. Usually the animal is a vertebrate such as a primate, rodent, domestic animal or game animal. The terms, “patient”, “individual” and “subject” are used interchangeably herein.

As used herein, the term “prolonged action” refers to long acting compositions, that is, compositions that have pharmacokinetic characteristics such that the composition provides for an extended length of release time than is normally found for the released drug (e.g. antitumor) itself.

As used herein, the term “pharmacologically acceptable carrier” is synonymous with “pharmacological carrier” and refers to any carrier that has substantially no long term or permanent detrimental effect when administered to subjects including humans and encompasses terms such as “pharmacologically acceptable vehicle, stabilizer, diluent, additive, auxiliary, or excipient.” Any of a variety of pharmaceutically acceptable carriers are used including, without limitation, aqueous media such as, e.g., water, saline, and glycine and the like.

### Compositions of the Disclosure

The hyaluronic acid of the present disclosure is crosslinked with a small molecular compound. Crosslinking results in improved solubility of the small molecular compound relative to the solubility of the compound itself. Moreover, crosslinking the hyaluronic acid results in a gel structure having very good water solubility while also imparting improved resistance to degradation of the small molecular compound. In an embodiment, the cross-linking agent is a small molecular compound such as an antitumor agent (the terms “antitumor agent” and “anticancer agent” can be interchangeable throughout the present disclosure). In a further embodiment, the antitumor compound can be at least one member selected from the group consisting of azacitidine, imatinib, lenalidomide, etoposide, topotecan, irinotecan, letrozole, raloxifene, cyclophosphamide, mechlorethamine, carbazylquinone, melphalan, thiotepa, busulfan, nimustine, carmustine, procarbazine, dacarbazine, methotrexate, 6-mercaptopurine, 6-thioguanine, azathioprine, 5-fluorouracil, ftorafur, floxuridine, cytarabine, ancitabine, doxifluridine, actinomycinD, bleomycin, mitomycin, chromomycin A3, cinelbin A, aclacinomycin A, adriamycin, peplomycin, cisplatin, mitoxantrone, epirubicin, pirarubicin, vinblastine, vincristine, vindesine, carboplatin, estramustine phosphate, mitotane, porphyrin, paclitaxel and docetaxel. In a further embodiment, the antitumor compound can be a boron-containing compound. The boron-containing compound can be mercaptoundecahydrododecaborate (BSH) or p-boronophenylalanine (BPA). In yet a further embodiment of the present disclosure, the pharmaceutical preparation containing the boron-containing compound can be used in boron neutron capture therapy (BNCT).

In yet a further embodiment of the present disclosure, the crosslinking is performed in the presence of an additional amount of the antitumor agent such that it becomes trapped or impregnated within the crosslinked hyaluronic acid network. The crosslinked hyaluronic acid network serves as a vehicle providing for prolonged bioavailability of the active(s) such as the antitumor agent(s). In an embodiment of the present disclosure, the crosslinked hyaluronic acid is obtained by extrusion. The use of extruders as continuous reactors for processes such as polymerization, polymer modification or compatibilization of polymer blends, involves technologies that have gained in popularity. In the case of reactive extrusion, several organic reactions can be conducted in extruders, including polymerization, grafting, copolymer formation, molecular network formation, crosslinking, functionalization and controlled degradation. In an embodiment of the present disclosure, a co-rotating intermeshing twin screw extruder (TSE) can be used. One of the advantages of using extruders as continuous reactors resides in the process being substantially a solid state chemical process. Accordingly, there is no real need for solvents in the extrusion process; the extrusion process typically produces less reaction side products; the extrusion process usually provides good yields of desired product; and the extrusion process enables the production of solid products from insoluble and thermo-labile starting materials.

In an embodiment of the present disclosure, the antitumor agent may be suitably combined with another drug (or with 3, 4, 5, 6 or more drugs) if necessary and contained in one drug delivery vehicle for cancer therapy. The other drug includes, but is not limited to: another antitumor agent(s); central nervous system drugs (for example, a general anesthetic, a hypnotic/analgesic agent, an antianxiety drug, etc.); peripheral nerve drugs (for example, a skeletal muscle relaxant, a spasmolytic agent etc.); circulatory drugs (for example, a cardiotonic agent, an antiarrhythmic agent, a diuretic agent, a hypotensive agent, a vasoconstrictor, a vasodilator, a lipid lowering drug, other circulatory drugs); respiratory drugs (for example, a respiratory stimulant, an antitussive agent, an expectorant, an antitussive expectorant, a bronchodilator etc.); digestive drugs (for example, an antiemetic drug, an antiflatulent, a stomachic digestive drug, an antacid, other digestive drugs etc.); hormonal agents (for example, a hypophysis hormone, a salivary gland hormone, a thyroid hormone, a parathyroid hormone, an anabolic steroid hormone, an adrenal hormone, an androgenic hormone, a mixed hormone, other hormones etc.); vitamin preparations (for example, vitamin A, vitamin D, vitamin B, vitamin C, vitamin E, vitamin K, a mixed vitamin, other vitamins etc.); allergy drugs (for example, an antihistamine); antibiotic drugs (for example, a drug acting on Gram-positive bacteria or Gram-negative bacteria); and antiviral drugs.

In an embodiment, the total amount of hyaluronic acid present in the compositions according to the present disclosure can range from about 50.0% to about 99.5% w/w of the composition. In an embodiment, the total amount of hyaluronic acid can range from about 60.0% to about 90.0% w/w of the composition. In a further embodiment, the total amount of hyaluronic acid can range from about 70.0% to about 80.0% w/w of the composition.

In an embodiment, the total amount of antitumor agent present in the compositions according to the present disclosure can range from about 0.5% to about 50.0% w/w of the composition. In an embodiment, the total amount of antitumor agent can range from about 10% to about 40% w/w of the composition. In a further embodiment, the total amount of antitumor agent can range from about 20% to about 30% w/w of the composition.

CD44 is expressed in a large number of mammalian cell types. CD44 is a widely distributed cell surface glycoprotein whose principal ligand has been identified as hyaluronic acid (HA). CD44 is involved in cell proliferation, cell differentiation, cell migration, angiogenesis, presentation of cytokines, chemokynes, and growth factors to the corresponding receptors, and docking of proteases at the cell membrane, as well as in signaling for cell survival. All these biological properties are essential to the physiological activities of normal cells, but they are also associated with the pathologic activities of cancer cells. Experiments in animals have shown that targeting of CD44 by antibodies, antisense oligonucleotides, and CD44-soluble proteins markedly reduces the malignant activities of various neoplasms, stressing the therapeutic potential of anti-CD44 agents. Since CD44 and its variants are typically overexpressed in a variety of cancer cell lines, it is surmised that the hyaluronic acid compositions of the present disclosure constitute a suitable delivery system for delivering an intended drug specifically to a desired cell or tissue.

In an embodiment, the cross-linked hyaluronic acid compositions of the present disclosure bind selectively to a particular target site possessed by a cell/affected organ. The cross-linked hyaluronic acid compositions of the present disclosure have target specificity and better selectivity for a defined population of cells/organs(s). In an embodiment of the present disclosure, the target site is CD44. Although most of the drugs mentioned hereinabove show efficacy to some extent, their lack of selectivity for tumor cells over normal cells often leads to severe side effects. Furthermore, the emergence of drug resistance remains a significant problem in the treatment of many cancers. The central problem in cancer chemotherapy is the severe toxic side effects of anticancer drugs on healthy tissues. Invariably the side effects impose dose reduction, treatment delay, or discontinuance of therapy. The hyaluronic acid compositions of the present disclosure reduce the uptake of an active drug by normal healthy cells while enhancing the influx and retention of the drug in cancer cells or tissues. Furthermore, the targeted delivery of the hyaluronic acid compositions of the present disclosure improves the bioavailability of the active drug while maximizing its effect by the sustained release from the compositions.

In an embodiment of the present disclosure, hyaluronic acid is target binding specific and directs the drug delivery vehicle to the target/tumor site. Once bound to the target, the drug is slowly released and internalized by the target/tumor site. Intracellular release of the cytotoxic drug is accomplished by cellular enzymes, preferably enzymes expressed in tumor cells.

### Uses and Methods of the Disclosure

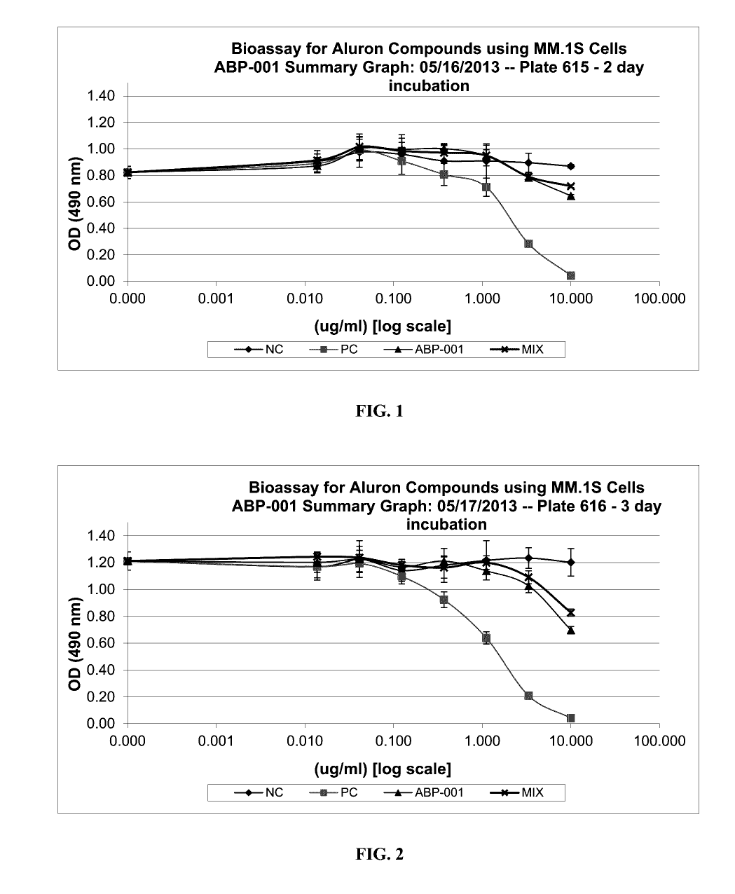

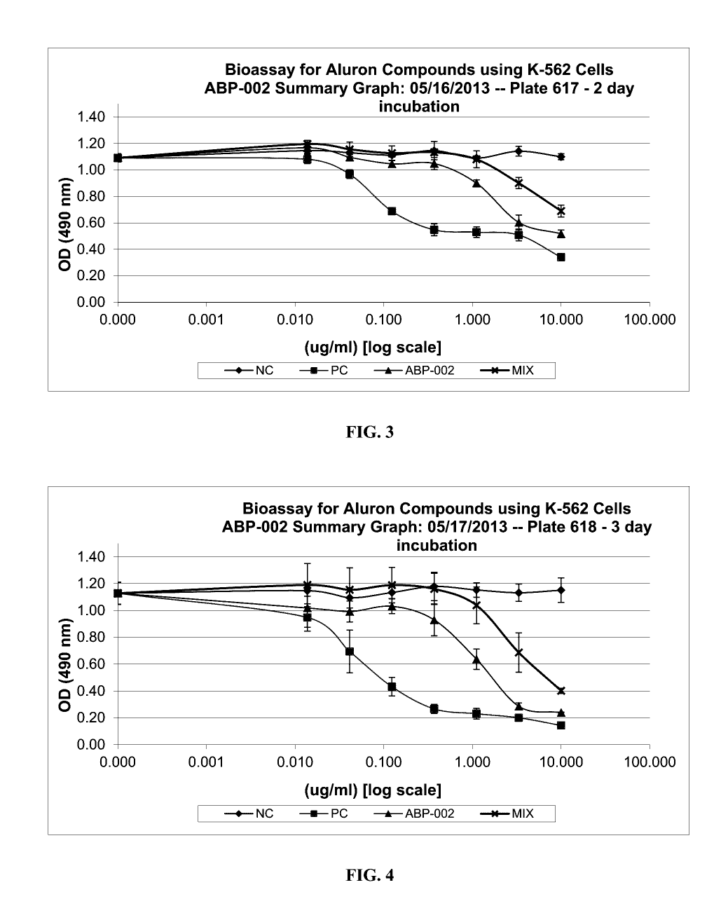

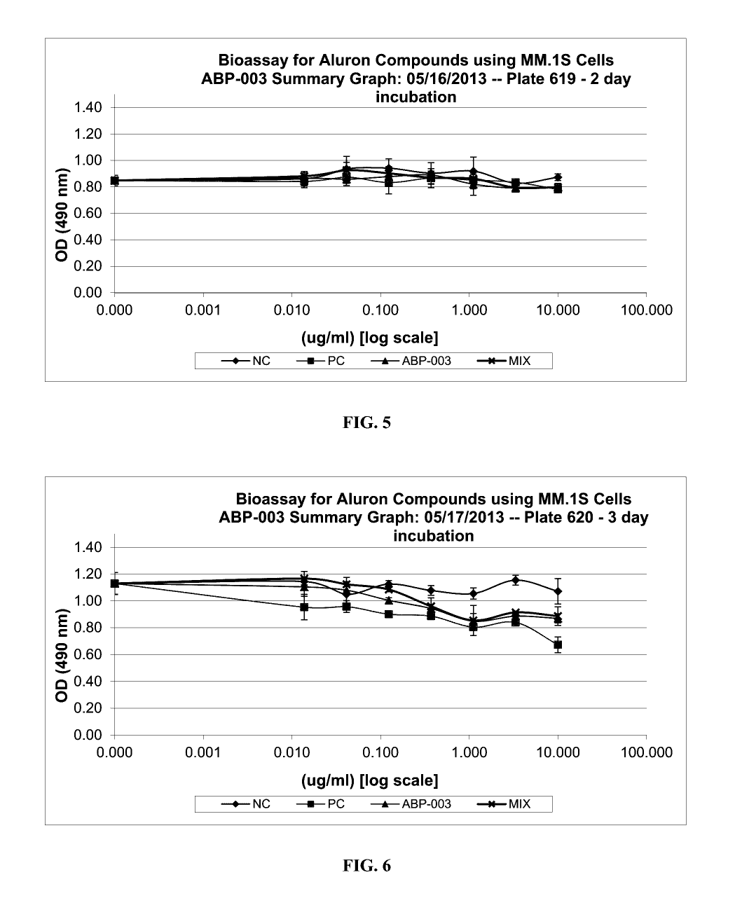

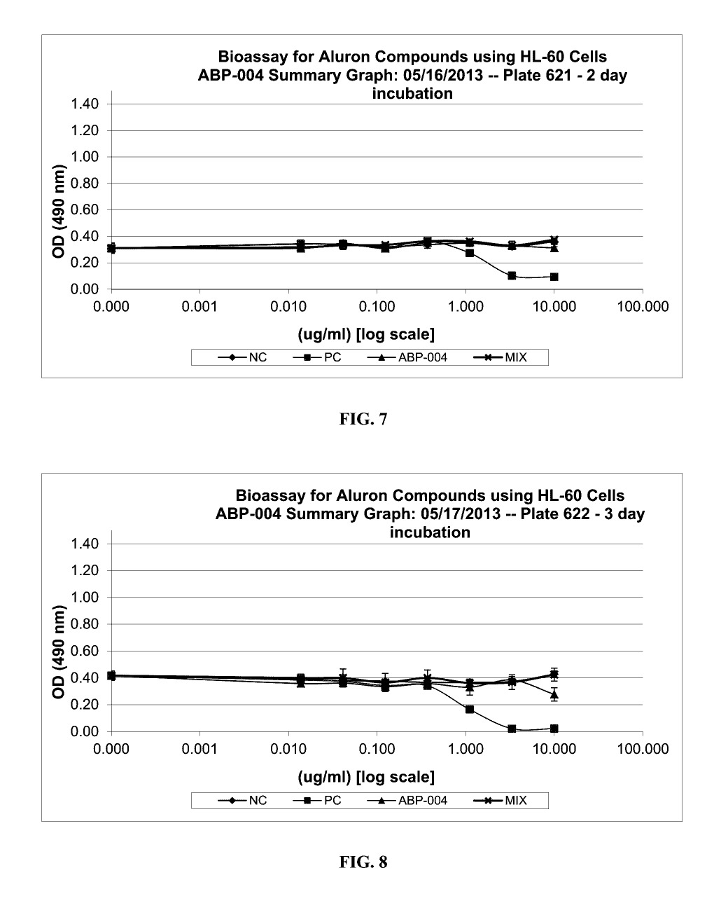

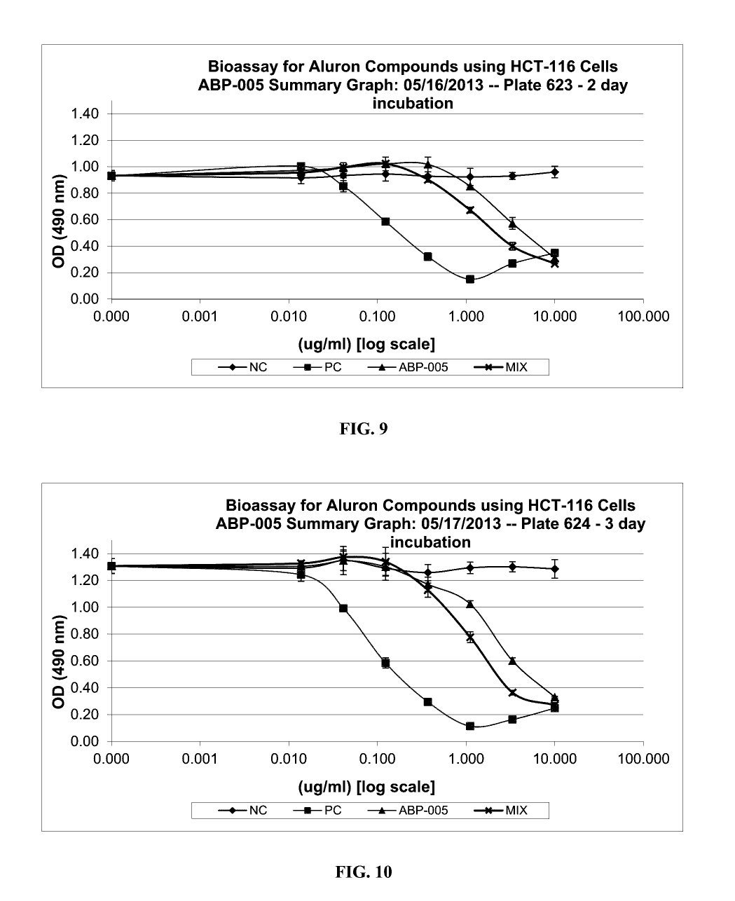

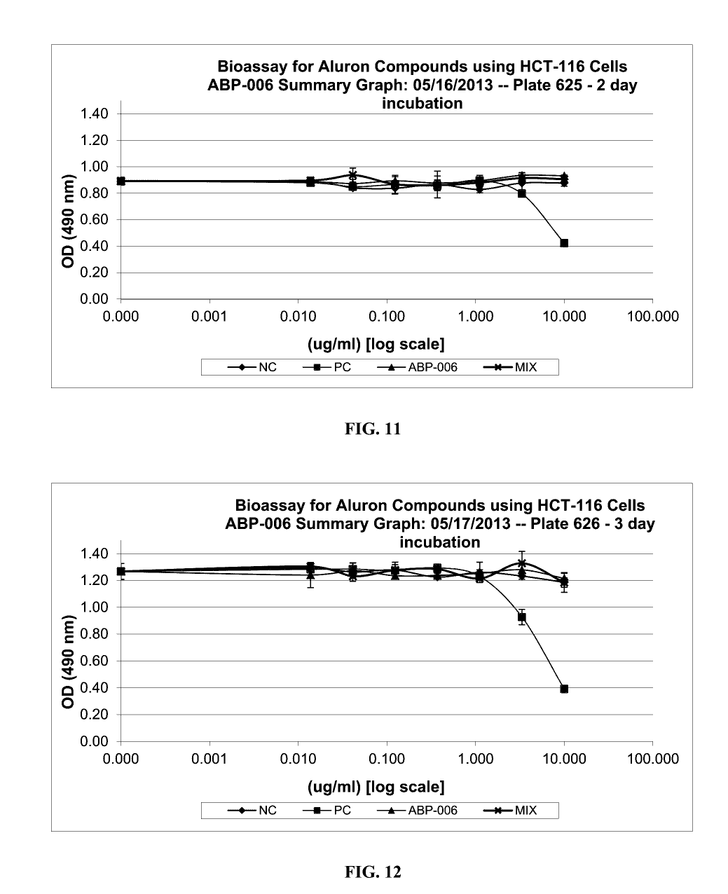

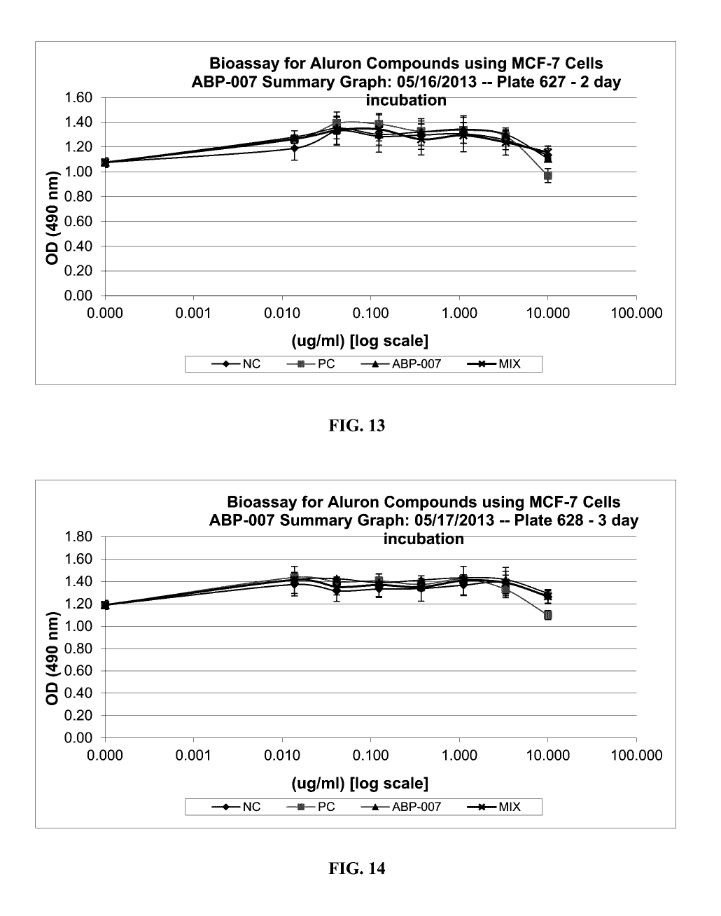

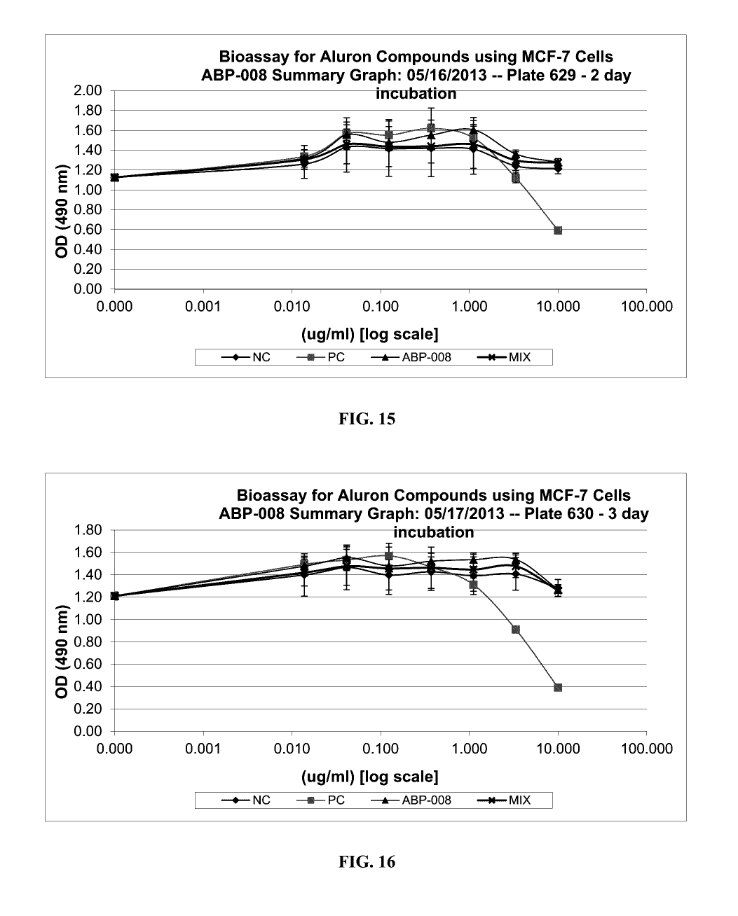

As illustrated in FIGS. 1-16, the effect of Azacitidine (HA-Azacitidine (9:1 w/w), FIGS. 1 and 2); Imatinib (HA-Imatinib (9:1 w/w); FIGS. 3 and 4); Lenalidomide (HA-Lenalidomide (9:1 w/w); FIGS. 5 and 6); Etoposide (HA-Etoposide (9:1 w/w); FIGS. 7 and 8); Topotecan (HA-Topotecan (9:1 w/w); FIGS. 9 and 10); Irinotecan (HA-Irinotecan (9:1 w/w); FIGS. 11 and 12); Letrozole (HA-Letrozole (9:1 w/w); FIGS. 13 and 14); and Raloxifene (HA-Raloxifene (9:1 w/w); FIGS. 15 and 16) on various cancer cell lines was studied. All hyaluronic-drug compositions were prepared using an extrusion process. The activities were measured in terms of overt cytotoxic effect following exposure for approximately two or three days. The negative control hyaluronic acid (HA) exhibited no overt cytotoxicity at doses up to 10 μg/ml on any of the 16 plates. Since HA is generally regarded as safe, an increase in activity (cytotoxicity) was not expected and none was observed. However, it has been shown that HA-complexed molecules (e.g crosslinked) may target CD-44 positive cells with a greater affinity than non-CD-44 positive cells, thus affording increased potency in a mixed population of normal and cancerous cells.

As illustrated in FIGS. 3-4 and 9-10, the HA-Imatinib and HA-topotecan complexes exhibited in vitro cytotoxicity and efficacy against the cancer cell lines in a dose and time dependent manner. A similar observation could be made for the HA-Azacitidine complex, but at higher doses (FIGS. 1-2). The HA-Lenalidomide complex (FIGS. 5-6) exhibited little or no inhibitory activity against MM.1S cells. The positive control and parent API exhibited potent 3 day cytotoxicity and inhibitory activity in HL-60 cells; however, the HA-Etoposide complex exhibited little activity.

In an embodiment of the present disclosure, the cross-linked hyaluronic acid serves as a drug delivery platform. In a further embodiment of the present disclosure, the hyaluronic acid provides a matrix improving the water solubility of the antitumor agent. In an embodiment of the present disclosure, the hyaluronic acid is crosslinked with the drug (e.g. antitumor agent) by at least one of covalent, ionic and/or electrostatic (e.g. H-bonding) interactions. The crosslinking provides for a stabilizing effect of the antitumor agent such that bioavailability of the antitumor agent is increased as well as improving the solubility thereof.

There are many poorly soluble drugs with very low bioavailability. Due to high affinity with water, the complexation of organic molecules (e.g. drugs) with HA creates gels having very high water solubility. In an aspect, the present disclosure relates to HA-drug complexes having high water solubility. It is surmised that these complexes impart increased bioavailability to the drug as compared to the parent drug alone. It is further surmised that lower drug concentrations (i.e. lower doses) can be used when the drug is in the form of a HA-drug complex resulting in lower observed toxicity and side effects while not downgrading the efficacy of the drug.

Due to the high affinity of HA to its protein receptor CD44, it is surmised, because of the observed over-expression of CD44 on tumor cells, that HA-drug complexes enable the targeted delivery of drug (e.g. antitumor agent) to tumor tissues and organs. Moreover, the targeted delivery provides the additional advantage of increased drug efficiency.

Several different HA-drug complexes where prepared and studied: HA-Irinotecan, HA-Raloxifene; and HA-Letrozole. Irinotecan exhibits good water solubility whereas both Raloxifene and Letrozole exhibit very poor water solubility. During the study, the Cₘₐₓ (maximum drug plasma concentration) and Tₘₐₓ (time required to reach Cₘₐₓ) values were measured. In the case of Irinotecan, the HA-Irinotecan complex did not show any significant improvement over the parent drug. This was not surprising given the good water solubility of Irinotecan (25 mg/ml). However, for Raloxifene, the HA-Raloxifene complex showed significant improvement for both Cₘₐₓ and Tₘₐₓ values when compared to the uncomplexed drug: Cₘₐₓ (μM) 0.84 and Tₘₐₓ (h) 3.5 (Table 6A) vs Cₘₐₓ (μM) 0.19 and Tₘₐₓ (h) 4.5. (Table 7A). A similar observation could be made for Letrozole, also showing significant improvement for both Cₘₐₓ and Tₘₐₓ values when compared to the uncomplexed drug: Cₘₐₓ (μM) 11.38 and Tₘₐₓ (h) 2.3 (Table 8A) vs Cₘₐₓ (μM) 4.24 and Tₘₐₓ (h) 4.5. (Table 9A). Similar observations were obtained with respect to concentrations in ng/mL (Tables 6B vs 7B and 8B vs 9B).

### Experimental

A number of examples are provided herein below illustrating the preparation of various cross-linked hyaluronic acid compositions in accordance with the present disclosure. The following non-limiting examples are illustrative of the present disclosure.

General Procedure for Preparing HA-Drug Complexes

In an embodiment of the present disclosure, HA-drug complexes (9:1 by weight) were prepared using a reactive extrusion process. In the reactive extrusion process, the drug becomes crosslinked to the HA matrix.

Hyaluronic acid was passed through an extruder to provide a post extruded hyaluronic acid. Aliquots of the post extruded hyaluronic acid were then mixed with drug (1:1), followed by further mixing in a mechanical mixer for a period of about two (2) hours. The composition was then passed through an extruder. In some embodiments of the present disclosure, the composition was passed through the extruder more than once. The resulting extruded composition was then mixed with additional post extruded hyaluronic acid and mixed in a mechanical mixer for about 2 hours. Finally, the composition was subjected to further extrusion.

Quantification of Drug Levels in Plasma Following p.o. Administration

The plasma drug levels of raloxifene, letrozole and irinotecan and its metabolite SN-38 were quantified following p.o. administration in male SD rats. Raloxifene was administered as a HA complex [HA-Raloxifene (HA-R)] or without HA [Raloxifene (R)] at 50 mg/kg (pure drug, HCl salt) in order to evaluate its pharmacokinetic parameters. Letrozole was administered as a HA complex [HA-Letrozole (HA-L)] or without HA [Letrozole (L)] at 5 mg/kg (pure drug, free base) in order to evaluate its pharmacokinetic parameters. Irinotecan was administered as a HA complex [HA-Irinotecan (HA-I)] or without HA [Irinotecan (I)] at 50 mg/kg (pure drug, HCl salt) in order to evaluate its pharmacokinetic parameters. Quantification was performed using the selective MRM mode on a short LC column using a fast gradient.

Materials

SD rat plasma K2-EDTA was purchased from BioreclamationIVT (Baltimore, Md., USA).

Equipment

Analytical balance Mettler Toledo (Model AT201); Eppendorf Microcentrifuge (Model 5424); LC/MS/MS AB/SCIEX 4000 QTRAP (Agilent 1100 series HPLC system consisting of an autosampler G1367A, column heater G1316A and binary pump G1312A). HPLC grade acetonitrile; ACS grade ammonium formate; ammonium acetate and formic acid (98%) were obtained from Fisher Scientific. Water was purified by a Milli-Q Synthesis A10 ultrapure water system from Millipore (Bedford, Mass., USA).

Animal and Sampling Protocol

In Vivo Protocol

For raloxifene, one group (6 animals per group) of male SD rats was administered a p.o. dose of HA-Raloxifene (HA-R) at 500 mg/kg, which corresponds to 50 mg/kg of pure drug (HCl salt), and one group (6 animals per group) was administered an oral dose of raloxifene at 50 mg/kg.

For letrozole, one group (6 animals per group) of male SD rats was administered a p.o. dose of HA-Letrozole (HA-L) at 50 mg/kg, which corresponds to 5.0 mg/kg of pure drug), and one group (6 animals per group) was administered an oral dose of letrozole at 5.0 mg/kg.

For irinotecan, one group (6 animals per group) of male SD rats was administered a p.o. dose of HA-Irinotecan (HA-I) at 500 mg/kg, which corresponds to 50 mg/kg of pure drug, HCl salt) and one group (6 animals per group) was administered an oral dose of Irinotecan at 50 mg/kg.

Blood samples were collected at time 0, 30 min and 1, 3, 6, 24, 30 and 48 hrs. All plasma samples were collected after centrifugation and frozen at −80° C. Plasma was transferred on dry ice to the Platform of Biopharmacy and was kept at −80° C. until extraction and LC/MS/MS analysis (Table 1).

TABLE 1
Summary of study in male SD rats.

| Compound | Dose¹ | Time points |
|---|---|---|
| HA-R | 50 mg/kg¹ | Predose, 30′, 1 h, 3 h, 6 h, 24 h, 30 h, 48 h. |
| R | 50 mg/kg | |
| HA-L | 5.0 mg/kg² | |
| L | 5.0 mg/kg | |
| HA-I | 50 mg/kg¹ | |
| I | 50 mg/kg | |

¹Equivalent to pure drug, HCl salt;
²Equivalent to pure drug.

Sample Analysis

Irinotecan and SN-38

The plasma tubes were thawed on ice, and kept on ice during the preparation. Plasma samples were vortex mixed and 40 μL and were pipetted into Eppendorf tubes. 10 uL of a 2M solution of NaF in water was rapidly added to minimize degradation of irinotecan by plasma esterase. Proteins were precipitated by the addition of 100 μL of internal standard (SN-22-0.5 μM in acetonitrile). The tubes were vortex mixed and centrifuged for 5 min at 13,000 rpm. 40 μL of supernatant was then transferred into a HPLC 96-well plate, and mixed with two volumes of water containing 5 mM ammonium formate at pH 4.0. The calibration curve was prepared in blank SD rat plasma, by serial dilution from 10 μM to 0.002 Standard plasma samples were treated as described above.

Raloxifene

Plasma samples were vortex mixed and 20 μL were pipetted into Eppendorf tubes. Proteins were precipitated by the addition of 40 μL of internal standard (labetalol—0.5 μM in acetonitrile). The tubes were vortex mixed and centrifuged for 5 min at 13,000 rpm. 40 μL of supernatant was transferred into a HPLC 96-well plate, and mixed with two volumes of water containing 0.2% formic acid. The calibration curve was prepared in blank SD rat plasma, by serial dilution from 10 μM to 0.002 Standard plasma samples were treated as described above.

Letrozole

The plasma tubes were thawed on ice. Plasma samples were vortex mixed and 20 μL were pipetted into Eppendorf tubes. Proteins were precipitated by the addition of 40 μL acetonitrile. The tubes were vortex mixed and centrifuged for 4.5 min at 13,000 rpm. 20 μL of supernatant was transferred into a HPLC 96-well plate, and mixed with two volumes of water containing 5 mM ammonium acetate. The calibration curve was prepared in blank SD rat plasma, by serial dilution from 10 μM to 0.002 Standard plasma samples were treated as described above.

Bioanalysis

Chromatography was performed on a Phenomenex Luna C8(2) 30×2 mm (5 μm) using gradient elution at 0.7 mL/min. The injection volume was 4 μL. The total acquisition time was 4.5 min. Samples were analyzed in selective MRM mode using LC-MS/MS. Peak area ratios for calibration curves and quantification of samples were calculated using Analyst software version 1.6.2. Calibration curves were plotted using the peak area ratios analyte/internal standard versus nominal analyte concentration, using a quadratic weighted 1/x regression.

TABLE 2A
Plasma concentrations of Irinotecan (μM) following an oral
dose of HA-Irinotecan at 500 mg/kg (equivalent to 50 mg/mL of
pure drug HCl salt) in male SD rats (group 34).

| | Group 34 Rat no # | | | | | | | |
|---|---|---|---|---|---|---|---|---|
| Time (hr) | 127 | 128 | 129 | 82 | 83 | 84 | Mean | SD |
| 0.5 | 0.008 | 0.049 | 0.007 | 0.008 | 0.028 | 0.002 | 0.017 | 0.018 |
| 1 | 0.007 | 0.036 | 0.008 | 0.016 | 0.021 | 0.004 | 0.015 | 0.012 |
| 3 | 0.014 | 0.043 | 0.008 | 0.102 | 0.105 | 0.010 | 0.047 | 0.046 |
| 6 | 0.008 | 0.018 | 0.120 | 0.075 | 0.030 | 0.003 | 0.042 | 0.046 |
| 24 | <LOQ | <LOQ | <LOQ | <LOQ | <LOQ | <LOQ | — | — |
| 30 | <LOQ | <LOQ | <LOQ | <LOQ | <LOQ | <LOQ | — | — |
| 48 | <LOQ | <LOQ | <LOQ | <LOQ | <LOQ | <LOQ | — | — |
| AUC_{0-inf} | 0.10 | 0.27 | 1.12 | 1.13 | 0.42 | 0.05 | 0.51 | 0.49 |
| [μM/h] | | | | | | | | |
| Cₘₐₓ (μM) | 0.014 | 0.049 | 0.120 | 0.102 | 0.105 | 0.010 | 0.067 | 0.049 |
| Tₘₐₓ (h) | 3 | 0.5 | 6 | 3 | 3 | 3 | 3.1 | 1.7 |
| C_{6 h} (μM) | 0.008 | 0.018 | 0.120 | 0.075 | 0.030 | 0.003 | 0.042 | 0.046 |

TABLE 2B
Plasma concentrations of Irinotecan (ng/mL) following an oral dose of
HA-Irinotecan 500 mg/kg (equivalent to 50 mg/mL of pure drug HCl salt)
in male SD rats (group 34).

| | Group 34 Rat no # | | | | | | | |
|---|---|---|---|---|---|---|---|---|
| Time (hr) | 127 | 128 | 129 | 82 | 83 | 84 | Mean | SD |
| 0.5 | 5.1 | 30.4 | 4.1 | 4.8 | 17.6 | 1.4 | 10.6 | 11.2 |
| 1 | 4.3 | 22.3 | 5.1 | 9.9 | 13.2 | 2.7 | 9.6 | 7.4 |
| 3 | 8.6 | 26.5 | 5.0 | 63.8 | 65.2 | 6.1 | 29.2 | 28.5 |
| 6 | 4.9 | 11.5 | 74.6 | 46.9 | 18.4 | 2.1 | 26.4 | 28.6 |
| 24 | <LOQ | <LOQ | <LOQ | <LOQ | <LOQ | <LOQ | — | — |
| 30 | <LOQ | <LOQ | <LOQ | <LOQ | <LOQ | <LOQ | — | — |
| 48 | <LOQ | <LOQ | <LOQ | <LOQ | <LOQ | <LOQ | — | — |
| AUC_{0-inf} | | | | | | | | |
| Cₘₐₓ (μM) | 8.6 | 30.4 | 74.6 | 63.8 | 65.2 | 6.1 | 41.5 | 30.4 |
| Tₘₐₓ (h) | 3.0 | 0.5 | 6.0 | 3.0 | 3.0 | 3.0 | 3.1 | 1.7 |
| C_{6 h} (μM) | 4.9 | 11.5 | 74.6 | 46.9 | 18.4 | 2.1 | 26.4 | 28.6 |

TABLE 3A
Plasma concentrations of Irinotecan (μM) following an oral dose of
Irinotecan at 50 mg/kg (pure drug HCl salt) in male SD rats (group 35).

| | Group 35 Rat no # | | | | | | | |
|---|---|---|---|---|---|---|---|---|
| Time (hr) | 133 | 134 | 135 | 88 | 89 | 90 | Mean | SD |
| 0.5 | 0.190 | 0.243 | 0.247 | 0.020 | 0.029 | 0.054 | 0.130 | 0.108 |
| 1 | 0.318 | 0.335 | 0.178 | 0.052 | 0.053 | 0.028 | 0.161 | 0.139 |
| 3 | 0.125 | 0.146 | 0.267 | 0.062 | 0.086 | 0.018 | 0.117 | 0.086 |
| 6 | 0.226 | 0.168 | 0.361 | 0.245 | 0.092 | 0.025 | 0.186 | 0.119 |
| 24 | 0.002 | 0.010 | 0.003 | 0.003 | 0.001 | 0.004 | 0.004 | 0.003 |
| 30 | 0.003 | 0.005 | 0.005 | 0.003 | 0.003 | 0.005 | 0.004 | 0.001 |
| 48 | <LOQ | <LOQ | <LOQ | <LOQ | <LOQ | <LOQ | — | — |
| AUC_{0-inf} | 3.23 | 2.84 | 4.87 | 2.86 | 1.31 | 0.51 | 2.60 | 1.53 |
| [μM * h] | | | | | | | | |
| Cₘₐₓ (μM) | 0.318 | 0.335 | 0.361 | 0.245 | 0.092 | 0.054 | 0.234 | 0.131 |
| Tₘₐₓ (h) | 1 | 1 | 6 | 6 | 6 | 0.5 | 3.417 | 2.836 |
| C_{30 h} (μM) | 0.003 | 0.005 | 0.005 | 0.003 | 0.003 | 0.005 | 0.004 | 0.001 |

TABLE 3B
Plasma concentrations of Irinotecan (ng/mL) following an oral dose of
Irinotecan at 50 mg/kg (pure drug HCl salt) in male SD rats (group 35).

| | Group 35 Rat no # | | | | | | | |
|---|---|---|---|---|---|---|---|---|
| Time (hr) | 133 | 134 | 135 | 88 | 89 | 90 | Mean | SD |
| 0.5 | 118.4 | 151.7 | 154.2 | 12.3 | 17.9 | 33.5 | 81.3 | 67.4 |
| 1 | 198.1 | 208.6 | 111.0 | 32.6 | 33.2 | 17.3 | 100.1 | 86.5 |
| 3 | 78.0 | 90.7 | 166.2 | 38.9 | 53.6 | 11.1 | 73.1 | 53.7 |
| 6 | 140.7 | 104.9 | 224.8 | 152.8 | 57.4 | 15.7 | 116.0 | 74.1 |
| 24 | 1.4 | 6.2 | 1.7 | 1.9 | 0.9 | 2.6 | 2.4 | 1.9 |
| 30 | 1.7 | 3.1 | 2.9 | 1.6 | 2.1 | 3.2 | 2.5 | 0.7 |
| 48 | <LOQ | <LOQ | <LOQ | <LOQ | <LOQ | <LOQ | — | — |
| AUC_{0-inf} | | | | | | | | |
| Cₘₐₓ (μM) | 198.1 | 208.6 | 224.8 | 152.8 | 57.4 | 33.5 | 145.9 | 81.7 |
| Tₘₐₓ (h) | 3.0 | 0.5 | 6.0 | 3.0 | 3.0 | 3.0 | 3.1 | 1.7 |
| C_{30 h} (μM) | 140.7 | 104.9 | 224.8 | 152.8 | 57.4 | 15.7 | 116.0 | 74.1 |

TABLE 4A
Plasma concentrations of SN-38 (μM) following an oral
dose of HA-Irinotecan at 500 mg/kg (equivalent to 50 mg/mL
of pure drug. HCl salt) in male SD rats (group 34).

| | Group 34 Rat no # | | | | | | | |
|---|---|---|---|---|---|---|---|---|
| Time (hr) | 127 | 128 | 129 | 82 | 83 | 84 | Mean | SD |
| 0.5 | 0.063 | 0.089 | 0.040 | 0.051 | 0.105 | 0.026 | 0.062 | 0.030 |
| 1 | 0.067 | 0.100 | 0.037 | 0.083 | 0.105 | 0.058 | 0.075 | 0.026 |
| 3 | 0.101 | 0.104 | 0.068 | 0.131 | 0.116 | 0.067 | 0.098 | 0.026 |
| 6 | 0.068 | 0.056 | 0.151 | 0.104 | 0.096 | 0.049 | 0.087 | 0.038 |
| 24 | <LOQ | <LOQ | <LOQ | <LOQ | <LOQ | <LOQ | — | — |
| 30 | <LOQ | <LOQ | <LOQ | <LOQ | <LOQ | <LOQ | — | — |
| 48 | <LOQ | <LOQ | <LOQ | <LOQ | <LOQ | <LOQ | — | — |
| AUC_{0-inf} | 0.98 | 0.78 | —¹⁾ | 1.92 | 2.17 | 0.81 | 1.33 | 0.66 |
| [μM * h] | | | | | | | | |
| Cₘₐₓ (μM) | 0.101 | 0.104 | 0.151 | 0.131 | 0.116 | 0.067 | 0.112 | 0.029 |
| Tₘₐₓ (h) | 3 | 0.5 | 6 | 3 | 3 | 3 | 3.1 | 1.7 |
| C_{6 h} (μM) | 0.068 | 0.056 | 0.151 | 0.104 | 0.096 | 0.049 | 0.087 | 0.038 |

¹Not possible to calculate AUC_{0-inf.}

TABLE 4B
Plasma concentrations of SN-38 (ng/mL) following an oral dose
of HA-Irinotecan at 500 mg/kg (equivalent to 50 mg/mL of pure drug
HCl salt) in male SD rats (group 34)

| | Group 34 Rat no # | | | | | | | |
|---|---|---|---|---|---|---|---|---|
| Time (hr) | 127 | 128 | 129 | 82 | 83 | 84 | Mean | SD |
| 0.5 | 24.9 | 34.8 | 15.5 | 19.9 | 41.2 | 10.1 | 24.4 | 11.8 |
| 1 | 26.2 | 39.1 | 14.6 | 32.7 | 41.3 | 22.6 | 29.4 | 10.2 |
| 3 | 39.5 | 40.8 | 26.8 | 51.6 | 45.5 | 26.2 | 38.4 | 10.1 |
| 6 | 26.6 | 22.0 | 59.3 | 40.6 | 37.8 | 19.3 | 34.3 | 14.9 |
| 24 | <LOQ | <LOQ | <LOQ | <LOQ | <LOQ | <LOQ | — | — |
| 30 | <LOQ | <LOQ | <LOQ | <LOQ | <LOQ | <LOQ | — | — |
| 48 | <LOQ | <LOQ | <LOQ | <LOQ | <LOQ | <LOQ | — | — |
| AUC_{0-inf} | | | | | | | | |
| Cₘₐₓ (μM) | 39.5 | 40.8 | 59.3 | 51.6 | 45.5 | 26.2 | 43.8 | 11.3 |
| Tₘₐₓ (h) | 3 | 1 | 6 | 3 | 3 | 3 | 3.1 | 1.7 |
| C_{6 h} (μM) | 26.6 | 22.0 | 59.3 | 40.6 | 37.8 | 19.3 | 34.3 | 14.9 |

TABLE 5
Plasma concentrations of SN-38 (μM) following an oral
dose of Irinotecan at 50 mg/kg (pure drug HCl salt) in male
SD rats (group 35).

| | Group 35 Rat no # | | | | | | | |
|---|---|---|---|---|---|---|---|---|
| Time (hr) | 133 | 134 | 135 | 88 | 89 | 90 | Mean | SD |
| 0.5 | 0.305 | 0.266 | 0.413 | 0.094 | 0.137 | 0.128 | 0.224 | 0.125 |
| 1 | 0.170 | 0.185 | 0.264 | 0.124 | 0.134 | 0.087 | 0.160 | 0.062 |
| 3 | 0.122 | 0.117 | 0.220 | 0.129 | 0.113 | 0.033 | 0.123 | 0.059 |
| 6 | 0.154 | 0.140 | 0.240 | 0.182 | 0.156 | 0.065 | 0.156 | 0.057 |
| 24 | 0.002 | 0.018 | 0.007 | 0.013 | 0.008 | 0.019 | 0.011 | 0.007 |
| 30 | 0.008 | 0.017 | 0.012 | 0.013 | 0.022 | 0.019 | 0.015 | 0.005 |
| 48 | <LOQ | <LOQ | <LOQ | <LOQ | <LOQ | <LOQ | — | — |
| AUC_{0-inf} | 2.34 | 2.55 | 3.76 | 2.72 | 2.37 | 1.48 | 2.54 | 0.74 |
| [μM * h] | | | | | | | | |
| Cₘₐₓ (μM) | 0.305 | 0.266 | 0.413 | 0.182 | 0.156 | 0.128 | 0.242 | 0.107 |
| Tₘₐₓ (h) | 0.5 | 0.5 | 0.5 | 6 | 6 | 0.5 | 2.3 | 2.8 |
| C_{30 h} (μM) | 0.008 | 0.017 | 0.012 | 0.013 | 0.022 | 0.019 | 0.015 | 0.005 |

TABLE 6A
Plasma concentrations of Raloxifene (μM) following an oral
dose of HA-Raloxifene at 500/kg (equivalent to 50 mg/mL of pure
drug HCl salt) in male SD rats (group 30).

| | Group 30 Rat no # | | | | | | | |
|---|---|---|---|---|---|---|---|---|
| Time (hr) | 79 | 80 | 81 | 85 | 86 | 87 | Mean | SD |
| 0.5 | 0.028 | 0.033 | 0.050 | 0.039 | 0.035 | 0.040 | 0.038 | 0.007 |
| 1 | 0.073 | 0.080 | 0.198 | 0.139 | 0.111 | 0.148 | 0.125 | 0.047 |
| 3 | 0.381 | 0.964 | 0.905 | 0.939 | 0.915 | 0.559 | 0.777 | 0.245 |
| 6 | 0.767 | 0.335 | 0.707 | 0.546 | 0.613 | 0.262 | 0.538 | 0.202 |
| 24 | 0.020 | 0.013 | 0.013 | 0.031 | 0.033 | 0.007 | 0.020 | 0.011 |
| 30 | 0.010 | 0.010 | 0.006 | 0.022 | 0.021 | 0.006 | 0.012 | 0.007 |
| 48 | <LOQ | 0.007 | <LOQ | 0.003 | 0.002 | 0.005 | — | — |
| AUC_{0-inf} | 9.44 | 6.44 | 10.15 | 8.95 | 9.56 | 4.58 | 8.19 | 2.19 |
| [μM * h] | | | | | | | | |
| Cₘₐₓ (μM) | 0.767 | 0.964 | 0.905 | 0.939 | 0.915 | 0.559 | 0.84 | 0.155 |
| Tₘₐₓ (h) | 6 | 3 | 3 | 3 | 3 | 3 | 3.5 | 1.2 |
| C_{30 h} (μM) | 0.010 | 0.010 | 0.006 | 0.022 | 0.021 | 0.006 | 0.012 | 0.007 |

TABLE 6B
Plasma concentrations of Raloxifene (ng/mL) following an
oral dose of HA-Raloxifene at 500 mg/kg (equivalent to 50 mg/kg
of pure drug HCl salt) in male SD rats (group 30).

| | Group 30 Rat no # | | | | | | | |
|---|---|---|---|---|---|---|---|---|
| Time (hr) | 79 | 80 | 81 | 85 | 86 | 87 | Mean | SD |
| 0.5 | 14.5 | 16.7 | 25.5 | 20.0 | 17.7 | 20.6 | 19.2 | 3.8 |
| 1 | 37.4 | 40.8 | 101.2 | 70.8 | 56.6 | 75.4 | 63.7 | 23.9 |
| 3 | 194.4 | 491.4 | 461.6 | 479.1 | 466.4 | 284.9 | 396.3 | 125.1 |
| 6 | 391.1 | 171.0 | 360.5 | 278.3 | 312.8 | 133.6 | 274.6 | 103.0 |
| 24 | 10.3 | 6.5 | 6.5 | 15.9 | 16.9 | 3.6 | 10.0 | 5.5 |
| 30 | 5.0 | 5.2 | 2.9 | 11.1 | 10.6 | 3.0 | 6.3 | 3.7 |
| 48 | <LOQ | 3.8 | 0.6 | 1.5 | 1.0 | 2.4 | 1.9 | 1.3 |
| AUC_{0-inf} | | | | | | | | |
| Cₘₐₓ (μM) | 391.1 | 491.4 | 461.6 | 479.1 | 466.4 | 284.9 | 429.1 | 78.8 |
| Tₘₐₓ (h) | 6.0 | 3.0 | 3.0 | 3.0 | 3.0 | 3.0 | 3.5 | 1.2 |
| C_{48 h} (μM) | <LOQ | 3.8 | 0.6 | 1.5 | 1.0 | 2.4 | 1.9 | 1.3 |

TABLE 7A
Plasma concentrations of Raloxifene (μM) following an oral dose of
Raloxifene at 50 mg/kg (pure drug HCl salt) in male SD rats
(group 31).

| | Group 31 Rat no # | | | | | | | |
|---|---|---|---|---|---|---|---|---|
| Time (hr) | 91 | 92 | 93 | 97 | 98 | 99 | Mean | SD |
| 0.5 | 0.012 | 0.009 | 0.023 | 0.019 | 0.030 | 0.010 | 0.017 | 0.009 |
| 1 | 0.023 | 0.007 | 0.028 | 0.021 | 0.048 | 0.013 | 0.023 | 0.014 |
| 3 | 0.094 | 0.081 | 0.324 | 0.151 | 0.363 | 0.062 | 0.179 | 0.131 |
| 6 | 0.087 | 0.095 | 0.249 | 0.180 | 0.244 | 0.082 | 0.156 | 0.079 |
| 24 | 0.006 | 0.015 | 0.003 | 0.004 | 0.022 | 0.002 | 0.009 | 0.008 |
| 30 | 0.007 | 0.005 | 0.003 | 0.003 | 0.017 | 0.008 | 0.007 | 0.005 |
| 48 | <LOQ | 0.067 | 0.003 | 0.013 | 0.003 | 0.025 | 0.022 | 0.027 |
| AUC_{0-inf} | 1.35 | 2.60 | 3.53 | 2.58 | 4.07 | 1.59 | 2.62 | 1.06 |
| [μM * h] | | | | | | | | |
| Cₘₐₓ (μM) | 0.094 | 0.095 | 0.324 | 0.180 | 0.363 | 0.082 | 0.190 | 0.125 |
| Tₘₐₓ (h) | 3 | 6 | 3 | 6 | 3 | 6 | 4.5 | 1.6 |
| C_{48 h} (μM) | <LOQ | 0.067 | 0.003 | 0.013 | 0.003 | 0.025 | 0.022 | 0.027 |

TABLE 7B
Plasma concentrations of Raloxifene (ng/mL) following an oral dose of
Raloxifene at 50 mg/kg (pure drug HCl salt) in male SD rats
(group 31).

| | Group 31 Rat no # | | | | | | | |
|---|---|---|---|---|---|---|---|---|
| Time (hr) | 91 | 92 | 93 | 97 | 98 | 99 | Mean | SD |
| 0.5 | 6.2 | 4.4 | 11.6 | 9.6 | 15.5 | 5.1 | 8.7 | 4.3 |
| 1 | 11.8 | 3.6 | 14.2 | 10.6 | 24.4 | 6.5 | 11.9 | 7.2 |
| 3 | 48.0 | 41.2 | 165.3 | 77.0 | 185.2 | 31.7 | 91.4 | 67.0 |
| 6 | 44.6 | 48.7 | 126.8 | 91.7 | 124.4 | 41.6 | 79.6 | 40.0 |
| 24 | 3.2 | 7.5 | 1.7 | 2.1 | 11.3 | 1.1 | 4.5 | 4.1 |
| 30 | 3.6 | 2.4 | 1.4 | 1.5 | 8.9 | 4.0 | 3.6 | 2.8 |
| 48 | <LOQ | 34.0 | 1.3 | 6.7 | 1.6 | 13.0 | 11.3 | 13.5 |
| AUC_{0-inf} | | | | | | | | |
| Cₘₐₓ (μM) | 48.0 | 48.7 | 165.3 | 91.7 | 185.2 | 41.6 | 96.8 | 63.7 |
| Tₘₐₓ (h) | 3 | 6 | 3 | 6 | 3 | 6 | 4.5 | 1.6 |
| C_{48 h} (μM) | <LOQ | 34.0 | 1.3 | 6.7 | 1.6 | 13.0 | 11.3 | 13.5 |

TABLE 8A
Plasma concentrations of Letrozole (μM) following an oral
dose of HA-Letrozole at 50 mg/kg (equivalent to 5.0 mg/kg of
pure drug) in male SD rats (group 32).

| | Group 32 Rat no # | | | | | | | |
|---|---|---|---|---|---|---|---|---|
| Time (hr) | 103 | 104 | 105 | 109 | 110 | 111 | Mean | SD |
| 0.5 | 10.09 | 8.48 | 7.58 | 7.24 | 8.86 | 9.93 | 8.70 | 1.17 |
| 1 | 10.74 | 9.70 | 9.16 | 9.30 | 14.04 | 10.42 | 10.56 | 1.81 |
| 3 | 10.39 | 10.19 | 11.58 | 10.44 | 10.03 | 11.30 | 10.66 | 0.63 |
| 6 | 8.846 | 8.528 | 8.445 | 9.355 | 9.557 | 7.192 | 8.65 | 0.84 |
| 24 | 6.956 | 6.411 | 6.108 | 8.496 | 9.113 | 5.100 | 7.03 | 1.51 |
| 30 | 6.826 | 6.378 | 6.418 | 8.512 | 7.597 | 6.556 | 7.05 | 0.85 |
| 48 | 6.095 | 4.527 | 4.832 | 5.864 | 4.572 | 4.592 | 5.08 | 0.71 |
| AUC_{0-inf} | 1429.3 | 616.7 | 760.1 | 748.5 | 548.8 | 882.2 | 831 | 316 |
| [μM * h] | | | | | | | | |
| Cₘₐₓ (μM) | 10.74 | 10.19 | 11.58 | 10.44 | 14.04 | 11.30 | 11.38 | 1.40 |
| Tₘₐₓ (h) | 1 | 3 | 3 | 3 | 1 | 3 | 2.3 | 1.0 |
| C_{24 h} (μM) | 6.095 | 4.527 | 4.832 | 5.864 | 4.572 | 4.592 | 5.080 | 0.708 |

TABLE 8B
Plasma concentrations of Letrozole (ng/mL) following an oral dose of
HA-Letrozole at 50 mg/kg (equivalent to 5.0 mg/kg of pure drug) in male
SD rats (group 32).

| | Group 32 Rat no # | | | | | | | |
|---|---|---|---|---|---|---|---|---|
| Time (hr) | 103 | 104 | 105 | 109 | 110 | 111 | Mean | SD |
| 0.5 | 2877 | 2419 | 2164 | 2065 | 2527 | 2833 | 2481 | 335 |
| 1 | 3065 | 2767 | 2612 | 2655 | 4005 | 2972 | 3013 | 517 |
| 3 | 2963 | 2908 | 3303 | 2979 | 2862 | 3225 | 3040 | 180 |
| 6 | 2524 | 2433 | 2409 | 2669 | 2727 | 2052 | 2469 | 240 |
| 24 | 1985 | 1829 | 1743 | 2424 | 2600 | 1455 | 2006 | 432 |
| 30 | 1947 | 1820 | 1831 | 2429 | 2167 | 1870 | 2011 | 242 |
| 48 | 1739 | 1291 | 1379 | 1673 | 1304 | 1310 | 1449 | 202 |
| AUC_{0-inf} | | | | | | | | |
| Cₘₐₓ (μM) | 3065 | 2908 | 3303 | 2979 | 4005 | 3225 | 3248 | 400 |
| Tₘₐₓ (h) | 1 | 3 | 3 | 3 | 1 | 3 | 2.3 | 1.0 |
| C_{24 h} (μM) | 1738.9 | 1291.5 | 1378.6 | 1673.1 | 1304.4 | 1310.0 | 1449 | 202 |

TABLE 9A
Plasma concentrations of Letrozole (μM) following an oral dose
of 5.0 mg/kg Letrozole in male SD rats (group 33).

| | Group 33 Rat no # | | | | | | | |
|---|---|---|---|---|---|---|---|---|
| Time (hr) | 115 | 116 | 117 | 121 | 122 | 123 | Mean | SD |
| 0.5 | 1.916 | 1.537 | 1.317 | 2.977 | 1.078 | 3.050 | 1.98 | 0.85 |
| 1 | 2.089 | 2.640 | 1.881 | 3.801 | 1.815 | 4.342 | 2.76 | 1.07 |
| 3 | 2.143 | 3.979 | 3.024 | 5.867 | 3.492 | 5.674 | 4.03 | 1.48 |
| 6 | 2.649 | 3.626 | 3.319 | 4.773 | 3.981 | 4.073 | 3.74 | 0.72 |
| 24 | 2.356 | 3.117 | 2.415 | 3.407 | 3.194 | 2.717 | 2.87 | 0.44 |
| 30 | 1.943 | 2.488 | 2.527 | 3.504 | 2.647 | 2.714 | 2.64 | 0.50 |
| 48 | 1.442 | 2.044 | 2.198 | 2.671 | 2.090 | 1.748 | 2.03 | 0.42 |
| AUC_{0-inf} | 175.0 | 265.1 | 580.9 | 414.9 | 268.1 | 233.3 | 323 | 149 |
| [μM * h] | | | | | | | | |
| Cₘₐₓ (μM) | 2.649 | 3.979 | 3.319 | 5.867 | 3.981 | 5.674 | 4.24 | 1.28 |
| Tₘₐₓ (h) | 6 | 3 | 6 | 3 | 6 | 3 | 4.5 | 6 |
| C_{24 h} (μM) | 1.442 | 2.044 | 2.198 | 2.671 | 2.090 | 1.748 | 2.03 | 0.42 |

TABLE 9B
Plasma concentrations of Letrozole (ng/mL) following
an oral of 5.0 mg/kg in male SD rats (group 33).

| | Group 33 Rat no # | | | | | | | |
|---|---|---|---|---|---|---|---|---|
| Time (hr) | 115 | 116 | 117 | 121 | 122 | 123 | Mean | SD |
| 0.5 | 546.7 | 438.4 | 375.6 | 849.3 | 307.5 | 870.2 | 565 | 242 |
| 1 | 595.9 | 753.2 | 536.7 | 1084.5 | 517.7 | 1238.9 | 788 | 305 |
| 3 | 611.4 | 1135.2 | 862.6 | 1673.9 | 996.3 | 1618.8 | 1150 | 422 |
| 6 | 755.8 | 1034.4 | 946.8 | 1361.7 | 1135.8 | 1161.9 | 1066 | 206 |
| 24 | 672.3 | 889.3 | 689.1 | 972.0 | 911.2 | 775.2 | 818 | 124 |
| 30 | 554.3 | 709.9 | 720.9 | 999.6 | 755.1 | 774.3 | 752 | 144 |
| 48 | 411.4 | 583.1 | 627.0 | 762.1 | 596.4 | 498.8 | 580 | 119 |
| AUC_{0-inf} | | | | | | | | |
| Cₘₐₓ (μM) | 755.8 | 1135.2 | 946.8 | 1673.9 | 1135.8 | 1618.8 | 1211 | 366 |
| Tₘₐₓ (h) | 6 | 3 | 6 | 3 | 6 | 3 | 4.5 | 1.6 |
| C_{24 h} (μM) | 411.4 | 583.1 | 627.0 | 762.1 | 596.4 | 498.8 | 580 | 119 |

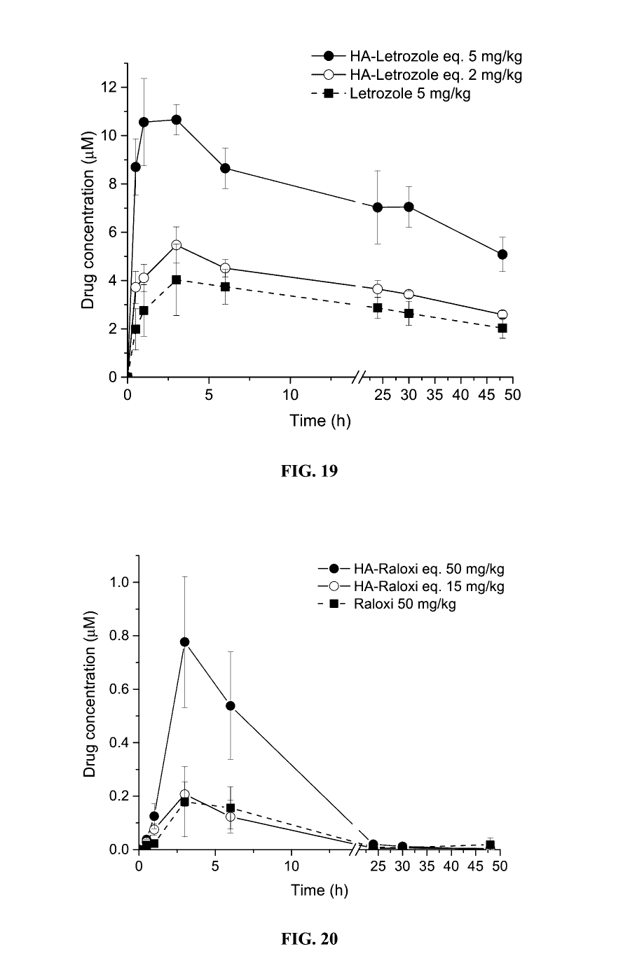

As illustrated in FIGS. 19 and 20, the bioavailability of Raloxifene and Letrozole is increased after combination with HA. Importantly, despite the fact that the bioavailability of Raloxifene and Letrozole is substantially increased, no detectable augmentation of the drug toxicity was found for either compound. Without wishing to be bound by theory, it is surmised that the improved bioavailability of the drug component is a result of crosslinking with HA leading to the formation of a colloid-like system upon dissolution of HA-drug complexes in water prior to administration.

Antitumor Activity of HA-Letrozole Complex in Female Crl:NU(NCr)-Foxn1^{nu} Nude Mice Bearing Human Breast MCF-7 Cells Following Twenty (20) Oral Administrations Over 4 Weeks.

HA-Letrozole complex (Aluron Biopharma Inc., Montreal, Qc, Canada). The content of Letrozole in the HA complexes corresponds to 10% by weight. Letrozole (98% pure) was obtained from ChemRF Laboratories Inc., Montréal, Québec, Canada). Carboxymethylcellulose sodium (CMC) was obtained from Sigma-Aldrich Inc., Oakville, Ontario, Canada).

Letrozole

Letrozole solutions were prepared once a week. Letrozole (15 mg) was reconstituted in 30.0 mL sterile water for injection to achieve a dose of 5 mg/kg (0.5 mg/mL). The reconstituted solution was vortexed every hour for ˜5 minutes for ˜8 hours followed by standing overnight at room temperature. The following day, the solution was sonicated for 10-20 minutes using a water bath sonicator filled with ice-cold water. Following dissolution, the Letrozole solution was aliquoted in 5 vials (for 5 day administrations) and stored at 4° C.

HA-Letrozole

Three (3) solutions of HA-Letrozole were prepared once a week: low dose (0.05 mg/mL), mid dose (0.5 mg/mL) and high dose (5.0 mg/mL). HA-Letrozole (150 mg) was reconstituted in 30 mL sterile water for injection to achieve a dose of 50 mg/kg (5 mg/mL). The reconstituted solutions were vortexed every hour for ˜5 minutes for ˜8 hours followed by standing overnight at room temperature. The next day the solutions were then finally vortexed for ˜1 minute and then aliquoted in 5 vials (for 5 day administrations) and stored at 4° C. The low dose HA-Letrozole solution at 0.5 mg/kg (0.05 mg/mL) was prepared by dilution of the 5 mg/kg solution with the appropriate volume of water for injection (1:10). The low dose HA-Letrozole solution was also aliquoted in 5 vials (for 5 administrations) and stored at 4° C. for administration.

CMC-Letrozole

A CMC-Letrozole solution was prepared once a week. The solution was prepared in three steps: 1) Letrozole (15 mg) was reconstituted in 30.0 mL sterile water for injection to achieve a dose of 5 mg/kg (0.5 mg/mL); 2) CMC (135 mg) was then mixed with the Letrozole solution; and 3) the reconstituted solution was vortexed every hour for ˜5 minutes for ˜8 hours followed by standing overnight at room temperature. The next day the solution was vortexed one last time and sonicated for 10-20 minutes using a water bath sonicator filled with ice-cold water. Following dissolution, the CMC-Letrozole solution was aliquoted in 5 vials (for 5 administrations) and stored at 4° C. The achieved dose was 50 mg/kg CMC-Letrozole (CMC 4.5 mg/mL+Letrozole 0.5 mg/mL).

Tumor Cell Line

The human MCF-7 breast cancer cell line was obtained from Sigma-Aldrich (ECACC; lot #12C002).

Animals

Seventy two (72) female Crl:NU(NCr)-foxn1nu nude mice (35-42 days) were obtained from Charles River Laboratories Inc., Wilmington, Mass., USA.

Tumor Inoculation

MCF-7 tumor was induced in 72 female Crl:NU(NCr)-foxn1nu nude mice by subcutaneous administration of the cancer cells in the left lower flank of each animal. Tumor cells were prepared in Matrigel® (Corning Life Sciences, Corning, N.Y., USA; Lot No. 4209014), a gelatinous protein mixture secreted by Engelbreth-Holm-Swarm (EHS) mouse sarcoma cells to imitate the complex extracellular environment found in many tissues and to promote the tumor growth (in absence of estrogen supplementation). Briefly, MCF-7 cells were trypsinized, centrifuged and washed once in sterile phosphate-buffered saline by centrifugation (400 g for 5 min. at room temperature). Cells were counted by the trypan blue exclusion test and the concentration adjusted in ice-cold sterile phosphate-buffered saline in order to get 1×10⁷ MCF-7 cells per 0.1 mL. Cells were then carefully mixed with Matrigel® (1:1) and kept on ice until injection. The animals were subcutaneously inoculated with 0.2 mL cells (1×10⁷ per mouse) in the left lower flank using a 25G needle. At the completion of the injection, the needle was turned 180° to prevent liquid (cells) leakage. Cell viability was determined at the end of the tumor inoculation by the trypan blue exclusion test.

Administration of the Test Articles (Treatment Schedule).

The dosing solutions were administered at twenty (20) occasions over 4 weeks by oral administration (gavage) using a 20G gavage needle. The dosing volume was 0.2 mL (˜10 mL/kg).

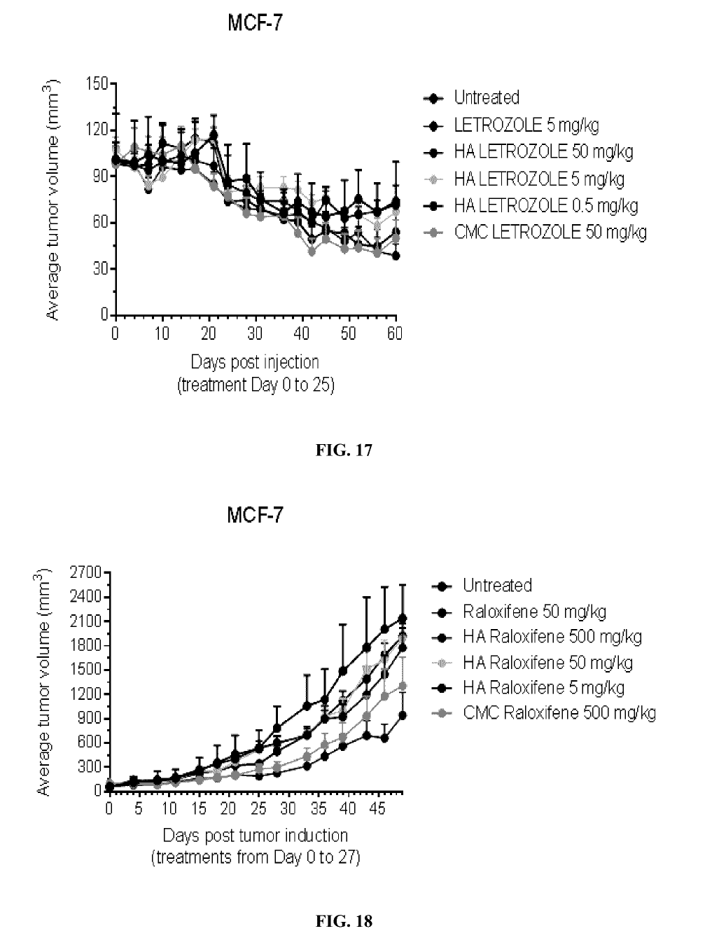

The mean tumor volume over time in the vehicle group and treated animals between days 0 to 60 following tumor implementation is illustrated in FIG. 17. Despite the low change in the tumor volumes, the highest dose of HA-Letrozole (50 mg/kg) showed to be significantly active towards human MCF-7 breast tumors (p=0.039) (Table 10). There is no dose-dependent relationship since HA-Letrozole at 5 mg/kg and 0.5 mg/kg were inactive (p=0.915 and p=0.570, respectively). Unconjugated Letrozole (free form) was also found to be inactive. CMC-Letrozole is barely active but the observed activity is not significant (Table 10). A tumor regression leading to tumor-free animals (no palpable masses) was observed for unconjugated letrozole (5 mg/kg), HA-Letrozole (50 mg/kg), HA-Letrozole (0.5 mg/kg) and CMC-Letrozole (50 mg/kg) (Table 11). Significant tumor regression was observed following repeated oral administrations of HA-Letrozole at the highest dose (50 mg/kg) (p=0.039), but not at lower doses (5 and 0.5 mg/kg). The minimal effective dose (MED) of HA-Letrozole showing a significant antitumor activity was therefore established at 50 mg/kg. The HA-Letrozole at 50 mg/kg corresponds to a dose of 5 mg/kg pure Letrozole. Pure Letrozole (5 mg/kg) was found to be inactive while CMC-Letrozole (50 mg/kg) was barely active, but the observed activity is not significant.

TABLE 10
Mean human breast MCF-7 tumor volumes and statistical analysis
(Student t-test) in the vehicle group and treated mice
at the end of the study (Day 60).

| | | Statistical analysis: | Statistical |
|---|---|---|---|
| | Mean tumor volume | untreated vs treated | significance |
| Treatment | (mm³) ± SD | animals (p value)* | (p ≦ 0.05) |
| Untreated | 74.0 ± 8.2 | N/A | N/A |
| Letrozole | 54.0 ± 13.9 | 0.258 | no |
| 5 mg/kg | | | |
| HA-Letrozole | 38.7 ± 7.5 | 0.039 | yes |
| 50 mg/kg | | | |
| HA-Letrozole | 67.2 ± 6.0 | 0.915 | no |
| 5 mg/kg | | | |
| HA-Letrozole | 71.8 ± 12.7 | 0.570 | no |
| 0.5 mg/kg | | | |
| CMC-Letrozole | 50.0 ± 11.8 | 0.085 | no |
| 50 mg/kg | | | |

*as determined by GraphPad Prism version 6

TABLE 11
Number of tumor-free animals (no palpable masses) at the end of the
study and the day (starting) when no tumor is observable.

| | Number of tumor-free | Starting day where no tumor is |
|---|---|---|
| Treatment | animals | observable. |
| Untreated | 0/10 | N/A |
| Letrozole | 3/10 | 39, 52, 52 |
| 5 mg/kg | | |
| HA-Letrozole | 2/10 | 36, 56 |
| 50 mg/kg | | |
| HA-Letrozole | 0/9 | N/A |
| 5 mg/kg | | |
| HA-Letrozole | 1/10 | 60 |
| 0.5 mg/kg | | |
| CMC-Letrozole | 2/10 | 28, 39 |
| 50 mg/kg | | |

Antitumor Activity of HA-Raloxifene Complex in Female Crl:NU(NCr)-Foxn1^{nu} Nude Mice Bearing Human Breast MCF-7 Cells Supplemented with 17β-Estradiol Following Twenty (20) Oral Administrations Over 4 Weeks.

HA-Raloxifene complex (Aluron Biopharma Inc., Montreal, Qc, Canada). The content of Raloxifene in the HA complexes corresponds to 10% by weight. Raloxifene hydrochloride salt (98% pure) was obtained from ChemRF Laboratories Inc., Montréal, Québec, Canada). Carboxymethylcellulose sodium (CMC) was obtained from Sigma-Aldrich Inc., Oakville, Ontario, Canada).

Raloxifene

Raloxifene solutions were prepared once a week. Raloxifene (75 mg) was reconstituted in 15.0 mL sterile water for injection to achieve a dose of 50 mg/kg (5.0 mg/mL). The reconstituted solution was briefly vortexed and sonicated for 10-20 minutes using a water bath sonicator filled with ice-cold water. Following dissolution, the Raloxifene solution was aliquoted in 5 vials (for 5 day administrations) and stored at 4° C.

HA-Raloxifene

Three (3) solutions of HA-Raloxifene were prepared once a week: low dose (0.5 mg/mL), mid dose (5.0 mg/mL) and high dose (50.0 mg/mL). HA-Raloxifene (15 mg, 150 mg and 1 g) was reconstituted respectively in 30 mL, 50 mL and 20 mL sterile water for injection to achieve doses of 5 mg/kg (corresponds to 0.5 mg/mL of pure drug), 50 mg/kg (5.0 mg/mL) and 500 mg/kg (50 mg/mL). The reconstituted solutions were vortexed every hour for ˜5 minutes for ˜8 hours followed by standing overnight at room temperature. The next day the solutions were vortexed for ˜1 minute and then aliquoted in 5 vials (for 5 day administrations) and stored at 4° C.

CMC-Raloxifene

A CMC-Raloxifene solution was prepared once a week. The solution was prepared in three steps: 1) Raloxifene (100 mg) was reconstituted in 20.0 mL sterile water for injection to achieve a dose of 50 mg/kg (5.0 mg/mL)—the reconstituted solution was briefly vortexed and sonicated for 10-20 minutes using a water bath sonicator filled with ice-cold water; 2) CMC (900 mg) was then mixed with the Raloxifene solution using a 3-way Stopcock syringe (BD, Franklin Lakes, N.J., USA); and 3) the reconstituted solution was vortexed every hour for ˜5 minutes for ˜8 hours followed by standing overnight at room temperature. The next day the solution was vortexed one last time, aliquoted in 5 vials (for 5 administrations) and stored at 4° C. The achieved dose was 500 mg/kg CMC-Raloxifene (CMC 45 mg/mL+Raloxifene 5 mg/mL).

Tumor Cell Line

The human MCF-7 breast cancer cell line was obtained from Sigma-Aldrich (ECACC; lot #12C002). To support the growth of the estrogen-dependent MCF-7 tumor, a 0.72-mg 17β-estradiol 60-day release pellet (Innovative Research of America, Sarasota, Fla.) was implanted s.c. under isoflurane anesthesia using a 10G trochar on the side opposite to the tumor implant side of the mouse one day before tumor implantation.

Animals

Seventy two (72) female Crl:NU(NCr)-foxn1nu nude mice (35-42 days) were obtained from Charles River Laboratories Inc., Wilmington, Mass., USA.

Tumor Inoculation

MCF-7 tumor was induced in 72 female Crl:NU(NCr)-foxn1nu nude mice by subcutaneous administration of the cancer cells in the left lower flank of each animal. Briefly, MCF-7 cells were trypsinized, centrifuged and washed once in sterile phosphate-buffered saline by centrifugation (400 g for 5 min. at room temperature). Cells were counted by the trypan blue exclusion test and the concentration adjusted in sterile phosphate-buffered saline in order to get 1×10⁷ MCF-7 cells per 0.1 mL. The animals were subcutaneously inoculated with 0.1 mL cells (1×10⁷ per mouse) in the left lower flank using a 25G needle. At the completion of the injection, the needle was turned 180° to prevent liquid (cells) leakage. Cell viability was determined at the end of the tumor inoculation by the trypan blue exclusion test.

Administration of the Test Articles (Treatment Schedule).

The dosing solutions were administered at twenty (20) occasions over 4 weeks by oral administration (gavage) using a 20G gavage needle. The dosing volume was 0.2 mL (˜10 mL/kg).

The mean tumor volume over time in the vehicle group and treated animals between days 0 to 49 following tumor implementation is illustrated in FIG. 18. Only the highest dose of HA-Raloxifene (500 mg/kg) showed to be significantly active towards human MCF-7 breast tumors (p=0.0178) (Table 12). There is no dose-dependent relationship since HA-Raloxifene at 50 mg/kg and 5.0 mg/kg were inactive. Unconjugated Raloxifene (free form) was also found to be inactive. No tumor regression was observed in the animals treated with HA-Raloxifene at 500 mg/kg even if a significant anticancer activity was noted. However, HA-Raloxifene slowed down the tumor growth at that given regimen (20 administrations over 4 weeks) (FIG. 18). HA-Raloxifene at 500 mg/kg is highly active towards human breast MCF-7 tumors. The minimal effective dose (MED) of HA-Raloxifene showing a significant antitumor activity was established at 500 mg/kg since the two (2) other doses, 5 and 50 mg/kg, were inactive. The HA-Raloxifene at 500 mg/kg corresponds to a dose of 50 mg/kg Raloxifene. Pure Raloxifene did not suppress tumor growth corroborating literature data suggesting poor efficacy of the drug at 0.3, 3.0 mg/kg/day (Brady H., Desai S., Gayo-Fung L. M. et al. Effects of SP500263, a Novel, Potent Antiestrogen, on Breast Cancer Cells and in Xenograft Models. Cancer Res. 62 (2002) 1439-1442) and 13.8 mg/kg (27 mole/kg) (Okamoto Y., Liu X., Suzuki N., et al. Increased antitumor potential of the raloxifene prodrug, raloxifene diphosphate. Int. J. Cancer 122 (2008) 2142-2147) in the MCF-7 xenograft model in mice. However, the study did demonstrate that crosslinking with HA increases the bioavailability of Raloxifene, HA-Raloxifene being the only compound to be active. Finally, HA-Raloxifene at 500 mg/kg was found to be more potent than CMC-Raloxifene at 500 mg/kg.

TABLE 12
Mean human breast MCF-7 tumor volumes and statistical analysis
(Student t-test) in the vehicle group and treated
mice at the end of the study (Day 49).

| | Mean tumor | Statistical analysis: | Statistical |
|---|---|---|---|
| | volume | untreated vs treated | significance |
| Treatment | (mm³) ± SD | animals (p value)* | (p ≦ 0.05) |
| Untreated | 2140.6 ± 546.2 | N/A | N/A |
| Raloxifene | 1919.5 ± 104.1 | 0.480 | no |
| 50 mg/kg | | | |
| HA-Raloxifene | 942.2 ± 287.4 | 0.018 | yes |
| 500 mg/kg | | | |
| HA-Raloxifene | 1892.2 ± 257.3 | 0.524 | no |
| 50 mg/kg | | | |
| HA-Raloxifene | 1787.7 ± 300.1 | 0..406 | no |
| 5 mg/kg | | | |
| CMC-Raloxifene | 1303.5 ± 357.4 | 0.079 | no |
| 500 mg/kg | | | |

*as determined by GraphPad Prism version 6

A tumor regression leading to tumor-free animals (no palpable masses) was observed for unconjugated letrozole (5 mg/kg), HA-Letrozole (50 mg/kg), HA-Letrozole (0.5 mg/kg) and CMC-Letrozole (50 mg/kg) (Table 11). Significant tumor regression was observed following repeated oral administrations of HA-Letrozole at the highest dose (50 mg/kg) (p=0.039), but not at lower doses (5 and 0.5 mg/kg). The minimal effective dose (MED) of HA-Letrozole showing a significant antitumor activity was therefore established at 50 mg/kg. The HA-Letrozole at 50 mg/kg corresponds to a dose of 5 mg/kg pure Letrozole. Pure Letrozole (5 mg/kg) was found to be inactive while CMC-Letrozole (50 mg/kg) was barely active, but the observed activity is not significant.

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

1. A water-soluble gel polymer matrix comprising:
hyaluronic acid having a molecular weight between 10,000 Da and 7,000,000 Da; and
an antitumor agent;
wherein the antitumor agent is crosslinked with the polymer matrix and wherein the polymer matrix improves the water solubility of the antitumor agent.

2. The water-soluble gel polymer matrix of claim 1, wherein the antitumor agent is crosslinked by at least one of covalent and/or electrostatic bonding.

3. The water-soluble gel polymer matrix of claim 2, wherein the antitumor agent is crosslinked by electrostatic bonding, and wherein the electrostatic bonding is hydrogen bonding.

4. The water-soluble gel polymer matrix of claim 2, wherein the antitumor agent is crosslinked by covalent bonding.

5. The water-soluble gel polymer matrix of claim 1, wherein the antitumor agent is selected from azacitidine, imatinib, lenalidomide, etoposide, topotecan, irinotecan, letrozole, raloxifene, cyclophosphamide, mechlorethamine, carbazylquinone, melphalan, thiotepa, busulfan, nimustine, carmustine, procarbazine, dacarbazine, methotrexate, 6-mercaptopurine, 6-thioguanine, azathioprine, 5-fluorouracil, ftorafur, floxuridine, cytarabine, ancitabine, doxifluridine, actinomycinD, bleomycin, mitomycin, chromomycin A3, cinelbin A, aclacinomycin A, adriamycin, peplomycin, cisplatin, mitoxantrone, epirubicin, pirarubicin, vinblastine, vincristine, vindesine, carboplatin, estramustine phosphate, mitotane, porphyrin, paclitaxel and docetaxel.

6. The water-soluble gel polymer matrix of claim 1, wherein the ratio of the hyaluronic acid to antitumor agent is from about 20:1 to about 2:1.

7. The water-soluble gel polymer matrix of claim 6, wherein the ratio of the hyaluronic acid to antitumor agent is from about 10:1 to about 2:1.

8. The water-soluble gel polymer matrix of claim 7, wherein the ratio of the hyaluronic acid to antitumor agent is from about 5:1 to about 2:1.

9. The water-soluble gel polymer matrix of claim 1, wherein the crosslinking is achieved by an extrusion process.

10. The water-soluble gel polymer matrix of claim 1, wherein the antitumor agent exhibits enhanced Cₘₐₓ (maximum drug plasma concentration) and Tₘₐₓ (time required to reach Cₘₐₓ) values.

11. A pharmaceutical delivery vehicle comprising:
hyaluronic acid having a molecular weight between 10,000 Da and 7,000,000 Da; and
an antitumor agent;
wherein the antitumor agent is crosslinked with the hyaluronic acid to form a water-soluble gel polymer matrix and wherein the polymer matrix improves the water solubility of the antitumor agent.

12. The pharmaceutical delivery vehicle of claim 11, wherein the antitumor agent is crosslinked by at least one of covalent and/or electrostatic bonding.

13. The pharmaceutical delivery vehicle of claim 12, wherein the antitumor agent is crosslinked by electrostatic bonding and wherein the electrostatic bonding is hydrogen bonding.

14. The water-soluble gel polymer matrix of claim 12, wherein the antitumor agent is crosslinked by covalent bonding.

15. The pharmaceutical delivery vehicle of claim 11, wherein the antitumor agent is selected from azacitidine, imatinib, lenalidomide, etoposide, topotecan, irinotecan, letrozole, raloxifene, cyclophosphamide, mechlorethamine, carbazylquinone, melphalan, thiotepa, busulfan, nimustine, carmustine, procarbazine, dacarbazine, methotrexate, 6-mercaptopurine, 6-thioguanine, azathioprine, 5-fluorouracil, ftorafur, floxuridine, cytarabine, ancitabine, doxifluridine, actinomycinD, bleomycin, mitomycin, chromomycin A3, cinelbin A, aclacinomycin A, adriamycin, peplomycin, cisplatin, mitoxantrone, epirubicin, pirarubicin, vinblastine, vincristine, vindesine, carboplatin, estramustine phosphate, mitotane, porphyrin, paclitaxel and docetaxel.

16. The pharmaceutical delivery vehicle of claim 11, wherein the ratio of the hyaluronic acid to antitumor agent is from about 20:1 to about 2:1.

17. The pharmaceutical delivery vehicle of claim 16, wherein the ratio of the hyaluronic acid to antitumor agent is from about 10:1 to about 2:1.

18. The pharmaceutical delivery vehicle of claim 17, wherein the ratio of the hyaluronic acid to antitumor agent is from about 5:1 to about 2:1.

19. The pharmaceutical delivery vehicle of claim 11, wherein the crosslinking is achieved by an extrusion process.

20. The pharmaceutical delivery vehicle of claim 11, wherein the antitumor agent exhibits enhanced Cₘₐₓ (maximum drug plasma concentration) and Tₘₐₓ (time required to reach Cₘₐₓ) values.

21. A process for preparing a crosslinked hyaluronic acid matrix, the method comprising:
extruding hyaluronic acid to produce extruded hyaluronic acid;
mixing the extruded hyaluronic acid with an antitumor agent to produce a mixture; and
extruding the mixture to produce the crosslinked hyaluronic acid matrix.

22. The process of claim 21, further comprising mixing the crosslinked hyaluronic acid matrix with additional hyaluronic acid and extruding.

23. The process of claim 21, wherein the hyaluronic acid has a molecular weight between 10,000 Da and 7,000,000 Da.

24. The process of claim 21, wherein the antitumor agent is selected from azacitidine, imatinib, lenalidomide, etoposide, topotecan, irinotecan, letrozole, raloxifene, cyclophosphamide, mechlorethamine, carbazylquinone, melphalan, thiotepa, busulfan, nimustine, carmustine, procarbazine, dacarbazine, methotrexate, 6-mercaptopurine, 6-thioguanine, azathioprine, 5-fluorouracil, ftorafur, floxuridine, cytarabine, ancitabine, doxifluridine, actinomycinD, bleomycin, mitomycin, chromomycin A3, cinelbin A, aclacinomycin A, adriamycin, peplomycin, cisplatin, mitoxantrone, epirubicin, pirarubicin, vinblastine, vincristine, vindesine, carboplatin, estramustine phosphate, mitotane, porphyrin, paclitaxel and docetaxel.

25. The process of claim 21, wherein the ratio of the hyaluronic acid to antitumor agent is from about 20:1 to about 2:1.

26. The process of claim 25, wherein the ratio of the hyaluronic acid to antitumor agent is from about 10:1 to about 2:1.

27. The process of claim 26, wherein the ratio of the hyaluronic acid to antitumor agent is from about 5:1 to about 2:1.

28. The process of claim 21, wherein the antitumor agent is crosslinked by covalent and/or electrostatic bonding.

29. The process of claim 28, wherein the antitumor agent is crosslinked by electrostatic bonding, and wherein the electrostatic bonding is hydrogen bonding.

30. The process of claim 28, wherein the antitumor agent is crosslinked by covalent bonding.
